(19) United States
(12) Patent Application Publication
(10) Pub. No.: US 2024/0148457 A1
(43) Pub. Date: May 9, 2024

(54) SMART ROBOT-ASSISTED BRAIN & SPINE SURGICAL SYSTEM

(71) Applicants: John M. Abrahams, Scarsdale, NY (US); Jeffrey Shasho, Brooklyn, NY (US); Jonah Saltzman, Brooklyn, NY (US)

(72) Inventors: John M. Abrahams, Scarsdale, NY (US); Jeffrey Shasho, Brooklyn, NY (US); Jonah Saltzman, Brooklyn, NY (US)

(21) Appl. No.: 18/215,444

(22) Filed: Jun. 28, 2023

Related U.S. Application Data
(63) Continuation of application No. 16/738,636, filed on Jan. 9, 2020, now abandoned.
(60) Provisional application No. 62/790,843, filed on Jan. 10, 2019.

(51) Int. Cl. A61B 34/00; A61B 34/10; A61B 34/32; A61B 90/00

(52) U.S. Cl. CPC A61B 34/70; A61B 34/10; A61B 34/32; A61B 90/37; A61B 2034/105; A61B 2034/107; A61B 2034/2055

(57) ABSTRACT

The present invention teaches a robotically-assisted surgical system, method and apparatus capable of achieving access to areas of the brain both within and outside a direct line of sight. This is achieved through a relatively small skull access port that is of lesser invasion than some achieved in the prior art. A console spaced from a surgical patient is adapted to comfortably accommodate a surgeon, in a manner to achieve minimal stress during lengthy surgical procedures. Robotics interconnect the surgeon and surgical tools to be used, said tools being capable of entering and operating through a relatively small (20 mm) port in the patient's skull. Camera means comprising part of said surgical tools being interconnected to a monitor viewable by the surgeon. Means are provided which are controllable by the surgeon for gaining access to and manipulating portions of the patient's brain both within a direct line of sight and outside of said direct line of sight.

This application claims the benefit of priority pursuant to 35 U.S.C. 119(e) from a U.S. Provisional Patent Application having Application No. 62/790,843 filed Jan. 10, 2019, the text of which is fully incorporated by reference herein, as if repeated, below.

### BACKGROUND OF THE INVENTION

### Introduction

This provisional patent application is meant to serve as an enhanced invention disclosure and will be replaced by a non-provisional utility patent application within one year of this filing date. The non-provisional application will include far more detail than set forth herein.

The present invention teaches a novel smart robot-assisted surgical system as well as techniques for use in surgeries such as, by way of example only, those affecting the brain and the spine. This invention teaches unique robotic methods and apparatus, as well as unique methods utilizing the robotic apparatus.

It should be noted that the use of the term “robotic surgery” in this specification of the present invention is not meant to suggest surgery performed autonomously by a robot without decision making and the presence and participation of a surgeon. The term “smart” is meant to denote robotics that are used to augment the activities of humans, not that which is guided by artificial intelligence, but which requires human involvement.

### Long Felt Need

There has been a long felt need for the type of novel apparatus and system offered to surgeons and their patients by the present invention. No longer will more skull be necessary to be removed. Multiple ports of entry may be unnecessary. Greater stability will be allowed. Greater visualization is possible. And the ability of robotics to permit access to areas of the brain previously unattainable will be possible. These and other objects of the invention will become apparent from a reading of this specification, taken in conjunction with the accompanying drawings.

### Prior & State of the Art

While the prior art neither discloses nor suggests the present invention and its novel capabilities, it is worthwhile examining the following representative examples of known surgical apparatus and techniques that have been suggested, published and/or performed by others in the past.

The use of robots and robotic apparatus in surgery, in general, is known. Advances aided by surgical robots have been minimally invasive surgery, remote-controlled surgery, and unmanned surgery. Robots permit precision, miniaturization, smaller incisions, decreased blood loss, less pain and more rapid healing times. This, in turn, enables reduced duration of hospital stays, blood loss, transfusions, and the use of pain medication. Robot-assisted surgery gives the surgeon better control of surgical instruments and a better view of the surgical site. Surgeons no longer tire from a need to stand throughout the surgical procedure. And naturally occurring hand tremors are filtered out by, for example, the robot's computer software.

For example, robotic stereotactic assistance (ROSA) is used to improve brain surgery procedures. ROSA utilizes an architecture that simulates movements of a human arm, thereby allowing the relatively rapid and precise placement of right temporal depth electrodes in performing stereo-electroencephalography (SEEG), in order to diagnose seizure onset zones in the brain.

Another example of the use of robotic assistance resides in a Bonn-based German-developed robotic-assisted cranial surgery, intended for use in the surgical therapy of craniosynostosis syndromes. These syndromes are characterized by premature fusions of cranial sutures, which have the potential to impair proper brain and craniofacial development. Computerized planning enables the position and shape of the intended craniotomy on a virtual model of the patient's skull. Thereafter, after removal of soft covering tissue, the robot autonomously performs the craniotomy.

As used in this specification, the term craniotomy means a surgery to cut a bony opening in the skull. Where the size of the bone flap is relatively small, the opening in the skull is sometimes referred to as a burr hole. Typically, a section of the skull, called a bone flap, is removed to access the brain. Craniotomies are sometimes named for the bone being removed, such as frontotemporal, parietal, temporal and suboccipital. A craniotomy is currently performed to treat brain tumors, hematomas (blood clots), aneurysms, traumatic head injury, foreign objects (bullets), swelling of the brain, and/or infection. Types of tumors removed may include meningiomas, pituitary tumors, craniopharyngioma, juvenile angiofibromas, chordomas, and esthesioneuroblastomas. The bone flap is usually replaced with tiny plates and screws once the procedure is completed. Where the bone flap is not replaced, the procedure is called a craniectomy.

Yet another example of prior art is the Intuitive Surgical American-made da Vinci surgical system. Approved by the FDA in 2000, this system attempts to improve upon conventional laparoscopy, and facilitates relatively complex surgeries utilizing minimal invasion—controlled by a surgeon who is located during the procedure at a comfortable console and monitor nearby in the same room as the patient. The costly da Vinci robotic-assisted system is not, however, known to be successfully used in brain or spine surgery, but rather is more commonly used for hysterectomies, prostatectomies, and cardiac valve repair, as well as a number of other procedures. Three da Vinci arms are manipulated by the surgeon and utilize tools that hold objects and can serve as scalpels, scissors, bovies, or graspers. When utilized successfully, the da Vinci system enables shorter hospital stays and more rapid recovery. That said, the da Vinci system is not without criticism.

A robotic-assisted system that was a rival to the da Vinci system, the Zeus robotic surgical system (ZRSS), was discontinued in 2003. Produced by the American company Computer Motion, the ZRSS system also had three robotic arms that were controlled remotely by a surgeon.

Other prior published references, while not anticipatory or suggestive of the present invention, which have been obtained from the Internet, include the following:
“Robots as surgical enablers”. MarketWatch. 3 Feb. 2005; “Prepping Robots to Perform Surgery”. The New York Times. 4 May 2008; “Company—Past Present Future”. Intuitive Surgical; “Surgical robots: The kindness of strangers”. The Economist. 21 Feb. 2013; “da Vinci Products”. Intuitive Surgical. 7 Apr. 2017; “The Slow Rise of the Robot Surgeon”. MIT Technology Review. 24 Mar. 2010; “da Vinci Robot Allegedly Marketed to Less-Skilled Doctors” Lawyers and Settlements.com. 23 Apr. 2013; “A comparison of total laparoscopic hysterectomy to robotically assisted hysterectomy: surgical outcomes in a community practice” J. Minim. Invasive Gynecol” 10.1016.2008.01.008; “Surgical Specialties—Regulatory Clearance”. Intuitive Surgical. Archived from the original on 16 Jan. 2013; “Robot Does Quick Fix on Prostate; interview with Dr Michael Palese”—(25 Jun. 2006). New York Daily News; “Transatlantic robot-assisted telesurgery”. Nature. 413 (6854): 379-380. doi:10.1038:35096636 via www.nature.com; “Salesmen in the Surgical Suite”. The New York Times. 25 Mar. 2013; “Patients Scarred After Robotic Surgery”—CNBC. 19 Apr. 2013; “Questions About Robotic Hysterectomy”. The New York Times. 25 Feb. 2013; “Robotically Assisted vs Laparoscopic Hysterectomy Among Women With Benign Gynecologic Disease”. The Journal of the American Medical Association, (20 Feb. 2013); Semiotic Flesh. Information and the Human Body, Seattle, WA: University of Washington Press, 2002, pp. 28-51. 27 Oct. 2013.

### BRIEF SUMMARY OF THE INVENTION

By further way of background, snake or continuum robots can be broadly defined as being separated into two general categories, namely, those of extrinsic actuation and those of intrinsic actuation.

The movements generated by extrinsic actuated robots are often enabled via the use of cables, whereby an exoskeleton or backbone elastic structure is moved by means of flexible cylindrical cables in tension. The actuation of these cables occurs outside of the relevant structure, thereby lending its identification or characterization as extrinsic actuation.

The movements generated by intrinsic actuated robots occur via internal means or by the structure itself.

The present invention is novel and can be distinguished from these extrinsic and intrinsic robots in that the smart retractor our invention is actuated externally, while the elastic structure itself is being actuated. Thus, the present invention can be characterized as a hybrid form of continuum robot that combines features of both intrinsic and extrinsic actuation.

Unlike known continuum robots that utilize three dimensional architectures—often cylindrical—the smart retractor according to the present invention utilizes what is essentially a flat two-dimensional architecture to achieve very similar movement characteristics. Full three-dimensional realization of our invention occurs by the addition of an additional degree of freedom via a pivoting of the two-dimensional structure. This enables the reaching of a three-dimensional sphere of points within the skull of a patient.

The present invention teaches a tool that may exist in a suite of tools designed to robotically assist a neurosurgeon performing surgery on the brain or the spine. In a preferred embodiment of this invention, this surgery is capable of being performed through a relatively small (20 mm) port in the skull. With this invention, the surgeon no longer must be limited by working by line of sight with brain tissue to be removed, for example. Furthermore, with the present invention the surgeon no longer must be limited to, by hand, manually non-robotically adjust a malleable spatula to be used as a tool. This manual spatula adjustment, which must often be repeated several times, is imprecise and difficult and consumes valuable surgery time. Furthermore, relatively large openings in the skull must be made.

What will herein sometimes be referred to as a “smart retractor robotic tool (SRRT)” functions as a spatula movable within and outside the line of sight to retract the brain or a tumor during neurological and intracranial procedures requiring removal of a tumor, for example. The SRRT is capable of being used to physically form a boundary layer between healthy and diseased brain tissue. The SRRT facilitates far less invasive surgical procedures through its ability to control the shape and disposition of the spatula once inside the patient's skull.

In a preferred embodiment of this invention, the SRRT is able to assume the form of a snake or continuum robot having a continuously curving-enabled manipulator that enjoys five (5) degrees of freedom. A lumen or outer “tongue” assembly is capable of being manipulated to plunge, pitch, as well as rotate. The distal or second stage of the SRRT, referred to as an inner tongue assembly, is capable of being manipulated to telescope in and out of the lumen, and is further capable of pitch movement. It is this combination of possible movements that permits the SRRT to accomplish virtually all functions of current non-robotic malleable spatulas, as well as being continuously actuatable inside of the patient's skull. These features permit relatively complex surgical maneuvers with relatively more precision within more confined surgical environments.

It is contemplated that the SRRT may be supplemented or augmented in order to provide the surgeon with a relatively complete robotic assembly for use in neurological procedures. For example, the SRRT will work in tandem with another tool such as a grasper or scissor, or a scissor with cautery capabilities and a second suction or irrigation tool. The complete robotic assembly just described will be capable of a surgical workspace that may be located fifty millimeters (50 mm) below the surface of the patient's skull.

### BRIEF DESCRIPTION OF THE SEVERAL VIEWS OF THE DRAWING

The accompanying drawings visually illustrate to one skilled in the art a preferred embodiment of the present invention. One skilled in the art will be able understand and appreciate what this invention teaches. Other configurations and embodiments are contemplated and will come within the proper and lawful scope of the present invention.

Referring now in more detail to the drawings and illustrations, which will be understood by one skilled in the art:

FIG. 1 is a front orthographic elevational view of a tongue assembly according to the present invention;

FIG. 2 is a side orthographic elevational view of the tongue assembly of FIG. 1;

FIG. 3 is an isometric elevational view of the tongue assembly of FIG. 1;

FIG. 4 is an orthographic front elevational view of one of two mechanisms that drive linear actuation according to the present invention;

FIG. 5 is a top view of the mechanism depicted in FIG. 4;

FIG. 6 is an enlarged perspective view of a portion of the subject invention shown below in FIG. 97;

FIG. 7 is a longitudinal cross section of the distal tip of a tongue according to this invention;

FIG. 8 is an enlarged isometric detail of the tongue of FIG. 7;

FIG. 9 is a sectional view of that depicted in FIG. 4;

FIG. 10 is an isometric view of that which is depicted in FIG. 4;

FIG. 11 is a section view showing the before (in full line) and after (in dotted line) states of articulation of the inner and outer snakes according to the present invention;

FIG. 12 is a sectional view of that which is depicted in FIG. 11, with the inner snake extended from the outer snake;

FIG. 13 is a sectional view of that which is depicted in FIG. 11, illustrating curved articulation of the inner snake;

FIG. 14 is an enlarged isometric view of a smart retractor tongue according to this invention;

FIG. 15 is an illustration of modes of articulation according to the invention;

FIG. 16 is a top down sectional view of the mechanism of the invention that drives linear articulation;

FIG. 17 is an illustration of the relatively largest notional workspace possible to reach with the present invention, operating through a 20 mm port in the skull of a patient to undergo robotic brain surgery with the present invention, where “A” is a workspace model, “B) is a tongue assembly according to the invention, and “C” is a smart tool used by the brain surgeon.

FIG. 18 is an illustration of a workspace (shown in red) and the 20 mm access port in the patient's skull;

FIG. 19 is a two-dimensional schematic cross-sectional depiction of the boundary of a patient's brain work area achievable by the smart retractor of the present invention;

FIG. 20 is an illustration of two regions of the brain that can be separated by use of the smart retractor of this invention, these regions possibly being regions of healthy and diseased brain tissue;

FIG. 21 is a schematic depiction of the two “antagonistic” strips or tongues of the retractor of the present invention, where the tongues are shown with and without a relatively thin and flexible, but of relatively high tensile strength, tight-fitting plastic casing or sleeve around the spatula;

FIG. 22 is a cross-sectional depiction of the inner smart retractor substantially thin metal tongues disposed with a layer of Teflon sandwiched in between them, thereby facilitating locating the tongues spaced from the neutral axis of the Teflon, thereby illustrating that the farther a strip is located from the neutral axis, the larger the section modulus and hence a larger bending moment can be resisted;

FIG. 23 is a view depicting the upper housing, the housing, and the tongue assembly of the smart retractor of the present invention, where the specific thickness of the metal tongues is a preferred 0.004 inch of 18-8 stainless steel or nickel titanium in order to provide both sufficient strength and flexibility to operate successfully in achieving relatively small bending radii without creating yielding of the material through its reaching a plastic state of permanent deformation; and where replacement of the tongue assembly is enabled via removal of the upper housing in medical applications;

FIG. 24 is a three-dimensional cross-sectional depiction of the tongue assembly of the present invention, with component parts labelled;

FIG. 25 is an elongated cross-sectional depiction of the tongue assembly of the present invention, labelled with preferred dimensions and choice of materials;

FIG. 26 is a fanciful depiction of the brain of a patient with a tumor to be removed, and with the skull secured in a fixed position pre-surgery;

FIGS. 27 through 33 depict successive steps in robotic brain surgery possible utilizing the present invention, and wherein positioning of the smart retractor of this invention is depicted in articulated modes;

FIG. 34 is an isometric view of the linear actuating assembly mechanism that creates movement of the inner and outer snakes of the articulating tool, controlled by robotics according to the present invention;

FIG. 35 is an isometric view similar to FIG. 34 of the mechanism that creates movement of the inner and outer snakes, according to the present invention;

FIG. 36 is an illustration of components which make up the linear actuating assembly mechanism according to the present invention;

FIG. 37 is a photograph depicting the full assembly of the full mechanism comprising the linear actuating assembly mechanism, including a harness that mates with a computer platform that is part of this invention, that facilitates motion controls;

FIG. 38 is a photograph depicting the rear portions of the full assembly of FIG. 4;

FIG. 39 is a photographic closeup detail view of the retractor according to the present invention;

FIG. 40 is a photographic closeup detail side view of the retractor shown in FIG. 6;

FIG. 41 is a photograph of the components of the smart robotic system according to the present invention, including, without limitation, the smart retractor, the motor harnessing, the power supply, the four motor controllers, the connectors that interface with the computer, and the controller;

FIG. 42 is a photographic view of the hand-operated controller that is used to “drive” the system, as well as its associated mapping between the controller inputs and the expected system outputs, it being understood that other more sophisticated hand-operated controllers are contemplated as coming within the scope of the present invention;

FIG. 43 is a schematic view illustrating the expected outputs realized by using the controller of FIG. 9;

FIG. 44 is a view similar to FIG. 1, but labelled to identify carriages 1 and 2, as well as the curl and depth control realized by utilizing the smart retractor mechatronics according to the present invention;

FIG. 45 is a schematic view of those portions of the system of the present invention previously shown in FIGS. 4 and 5, but labelled to identify the upper and lower retractors and their coupling of depth and curve controls;

FIG. 46 is a photograph illustrating the bending of the outer portion of the snake according to the present invention, and further illustrating with human fingers the bending of the inner snake portion according to the present invention;

FIG. 47 is a schematic orthographic view of an illustration of a prototype concept of the present invention;

FIG. 48 is an enlarged schematic isometric view illustrating the relative disposition of the inner and outer snakes according to the present invention;

FIG. 49 is a schematic isometric view illustrating the driving mechanism associated with the prototype concept shown in FIG. 48;

FIG. 50 is a photograph of the mechanism shown in FIG. 49;

FIG. 51 is a photograph of a portion of the mechanism shown in FIG. 50;

FIG. 52 is a perspective view of a portion of the driving mechanism assembly of the present invention;

FIG. 53 is a photograph of the inner and outer snakes according to the present invention with the inner snake relatively fully retracted within the outer snake, and in an unbent configuration (Note: the outer and inner snakes according to the present invention are sometimes herein described as outer snake portions and inner snake portions, without departing from the spirit of the invention);

FIGS. 53 through 71 illustrate a progression of the manipulation of the inner snake with respect to the outer snake of the articulating tool controlled by robotics, where, according to the present invention—especially FIG. 19), the surgeon is able to access parts of the brain other than and in addition to those parts within a direct line of sight;

FIGS. 72 through 94 duplicate and comprise slides of a PowerPoint presentation meant to illustrate a patient on a table to undergo robotic surgery using the present invention illustrated in blue;

FIGS. 95 and 96 are further illustrations or graphic depictions of a patient's skull in cross section, showing the present invention disposed within the brain workspace, with tools 1 and 2 labelled, as is the workspace, the camera, and the smart retractor, where FIG. 95 is without labelling and FIG. 96 includes elements labelled; and

FIGS. 97 through 101 are enlarged perspective views of the tongue assembly and tools of the present invention, with the smart retractor, camera, and tools 1 and 2, where appropriate, labelled.

### DETAILED DESCRIPTION OF THE INVENTION

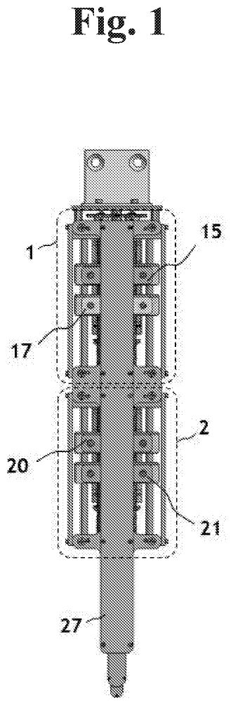

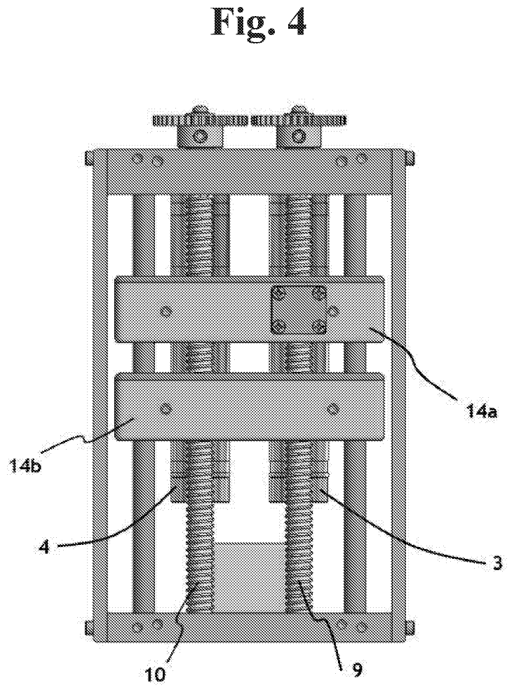

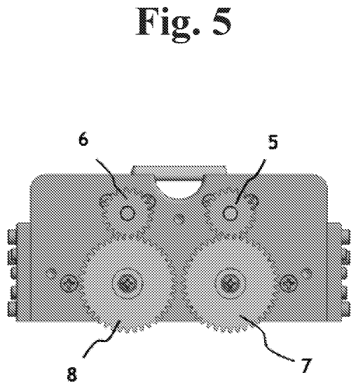

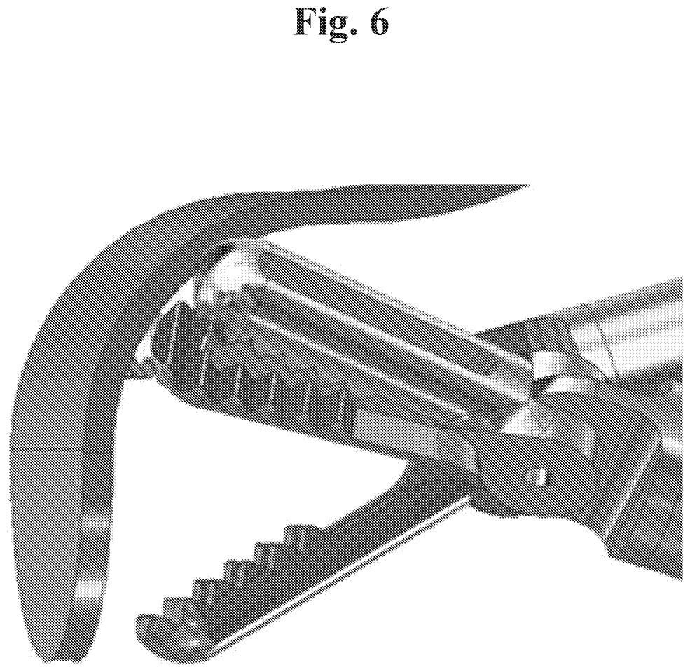

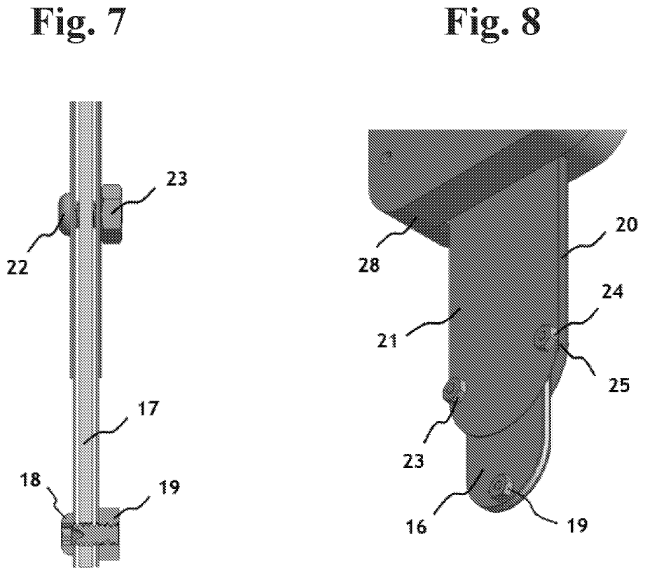

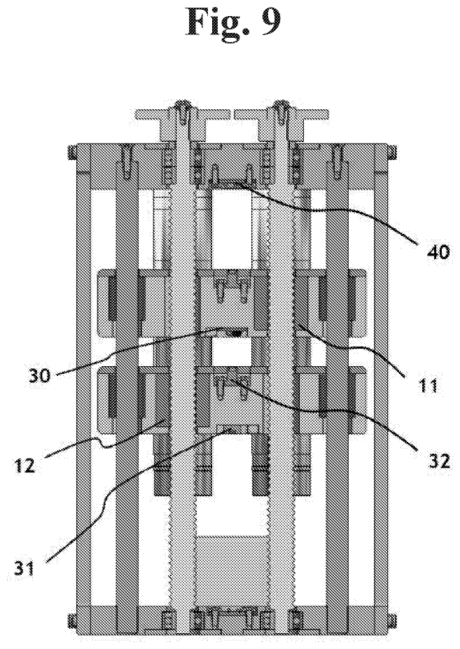

By way of example of a preferred embodiment of the invention, the robotic aspect of the present invention comprises a tongue subsystem & two separate drive units—an upper drive unit 1 and a lower drive unit 2 (FIG. 1). The upper drive unit has two motors, 3 and 4 (FIG. 4), that each drive a separate spur gear, 5 and 6 respectively (FIG. 5). Spur gear 5 drives spur gear 7 (FIG. 5) which is coupled to ball screw 9 (FIG. 4). Nut 11 (FIG. 8) is driven up and down ball screw 9 along with carriage 14a (FIG. 4). Carriage 14a is rigidly attached to tongue strip 15 (FIG. 1). Spur gear 6 drives spur gear 8 (FIG. 5) which is coupled to ball screw 10 (FIG. 4). Nut 12 (FIG. 9) is driven up and down ball screw 10 along with carriage 14b (FIG. 4). Carriage 14b is rigidly attached to tongue strip 16 and Teflon strip 17 (FIGS. 7 and 8). Teflon strip 17 is sandwiched by tongue strip 15 and tongue strip 16 and rigidly pinned by screw 18 and nut 19 (FIG. 7). The lower drive unit 2 is identical in form to the upper drive unit 1 except that drive unit 2 is attached to tongue strip 20 and tongue strip 21 (FIGS. 1 and 8). Tongue strips 20 and 21 are rigidly pinned by screws 22 and 25 with nuts 23 and 24 respectively (FIGS. 7 and 8). Thus, the inner tongue assembly formed by tongue strips 15 and 16 with Teflon strip 17 can move all together up and down between tongue strips 20 and 21.

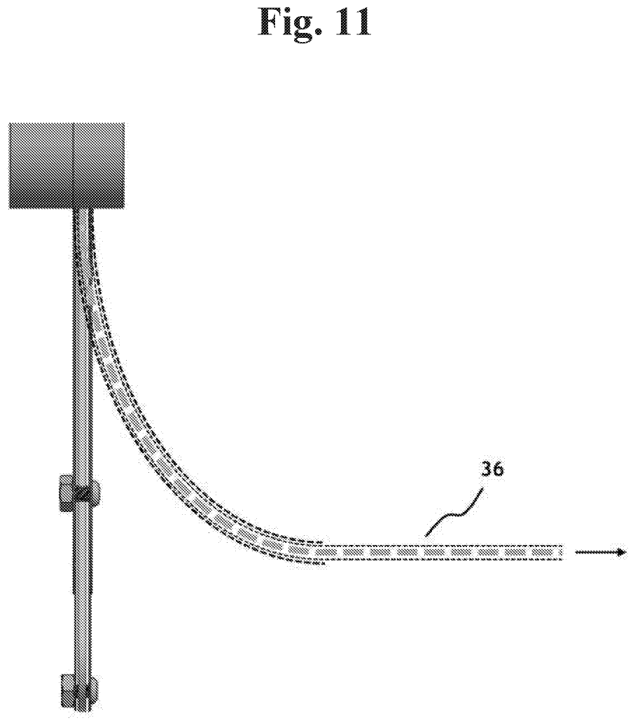

Forward bending actuation 35 of the outer tongue strip assembly formed by tongue strips 20 and 21 (FIG. 11) can be accomplished by either pulling up on tongue strip 20 by actuating carriage 14a of the lower drive unit 2 and keeping tongue strip 21 stationary or pushing down on tongue strip 21 by actuating carriage 14b of the lower carriage 2 and keeping tongue strip 20 stationary or a combination of pulling up on tongue strip 20 and pushing down on tongue strip 21. Backward bending actuation can be accomplished through opposite pushing and pulling respectively.

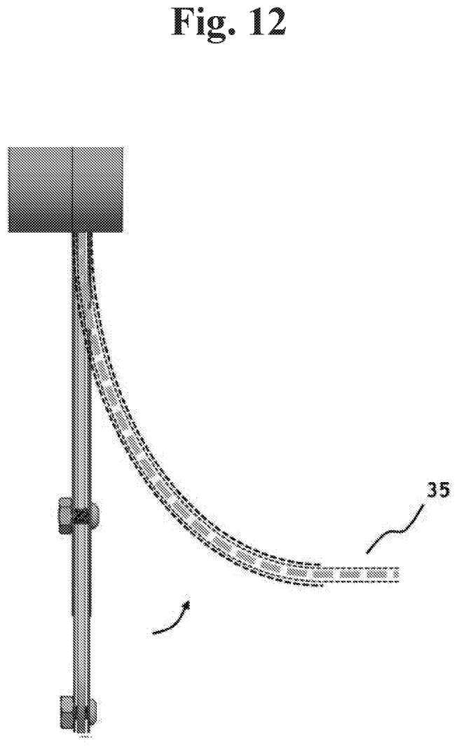

After actuation 35 is complete, translation of the inner tongue assembly through actuation 36 (FIG. 12) can be accomplished. Tongue strips 15 and 16 and Teflon strip 17 are all pushed by actuating both carriage 14a and 14b of the upper drive unit 1. This movement can be reversed by pulling on tongue strips 15 and 16 and Teflon strip 17 by actuating both carriage 14a and 14b of the upper drive unit 1.

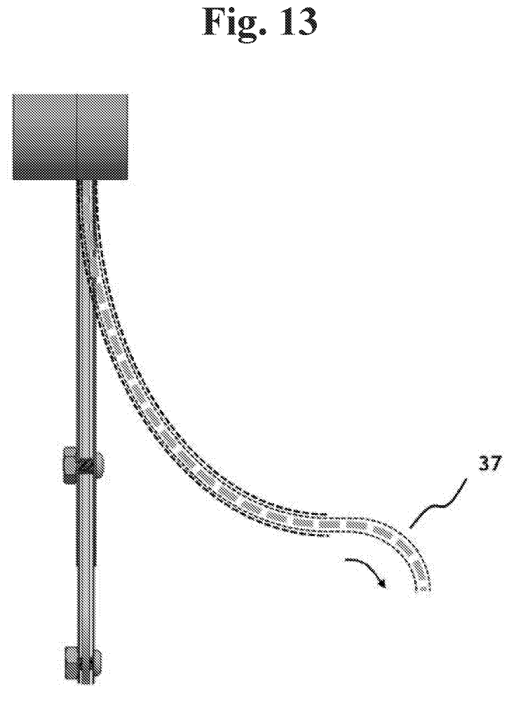

After actuation 36 is complete, downward bending actuation 37 (FIG. 13) of the inner tongue strip assembly can be accomplished. This is accomplished by either pulling up on tongue strip 15 by actuating carriage 14a of the upper drive unit 1 and keeping tongue strip 16 and Teflon strip 17 stationary, or by pushing down on tongue strip 16 and Teflon strip 17 by actuating carriage 14b of the upper drive unit 1 and keeping tongue strip 15 stationary or by pulling up on tongue strip 15 and pushing down on tongue strip 16 and Teflon strip 17. Both reversing this movement and upwards bending actuation can be accomplished through opposite pushing and pulling respectively.

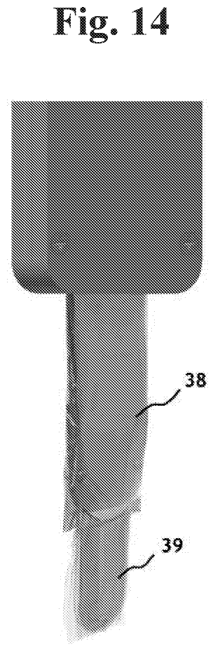

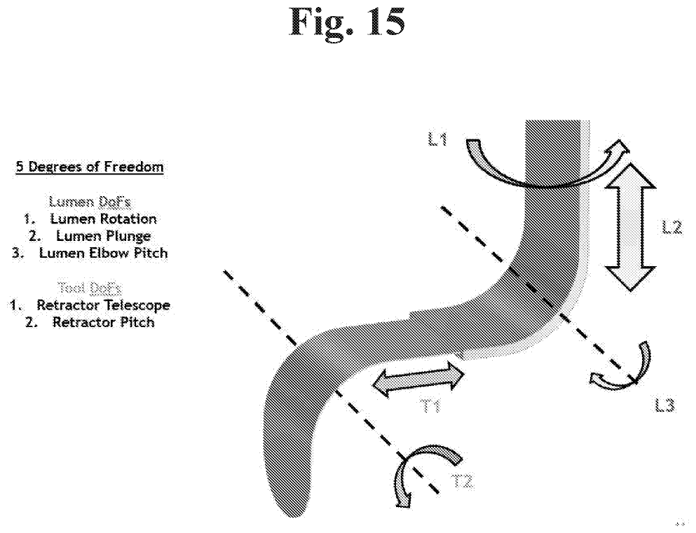

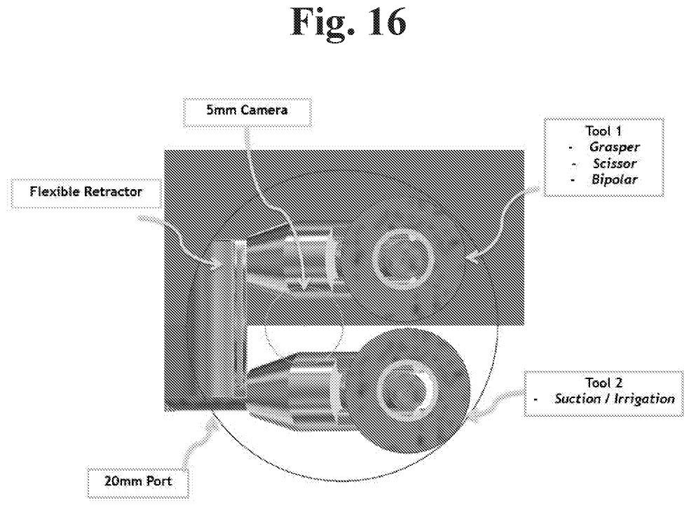

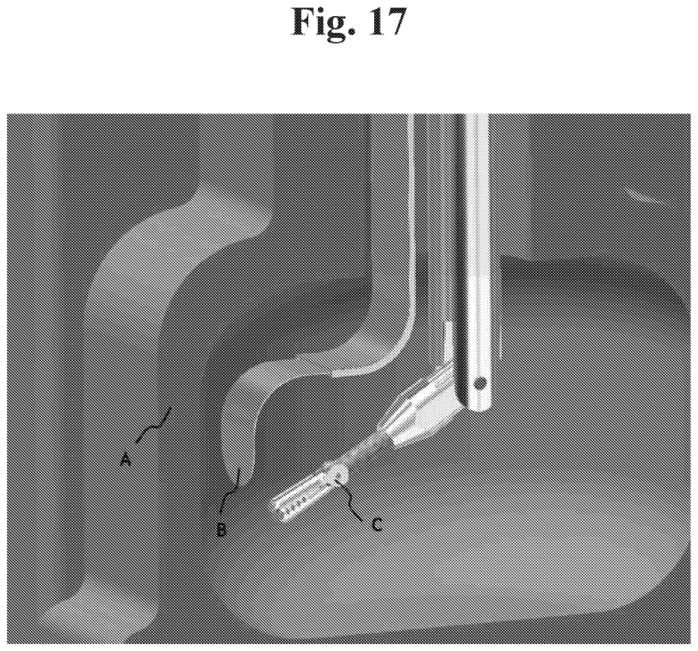

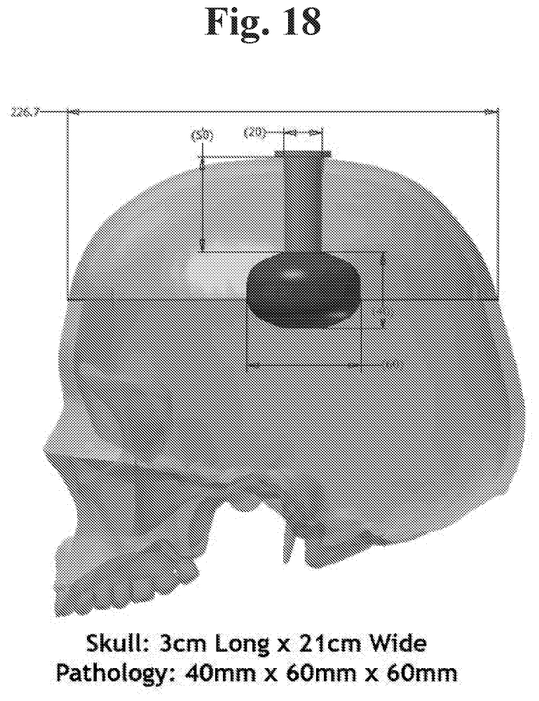

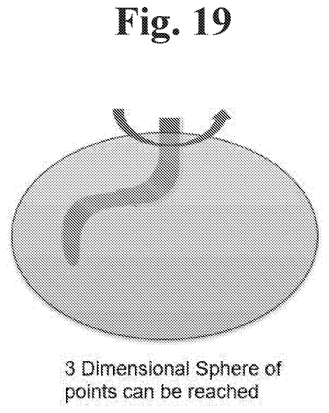

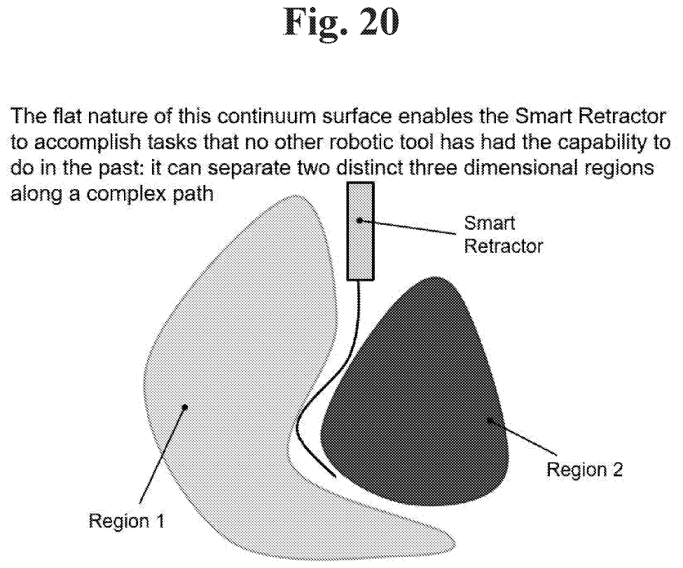

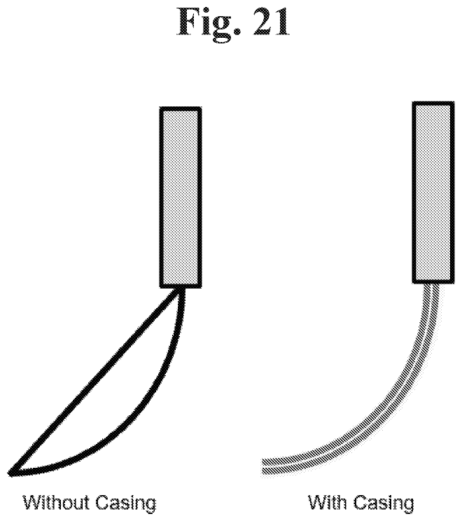

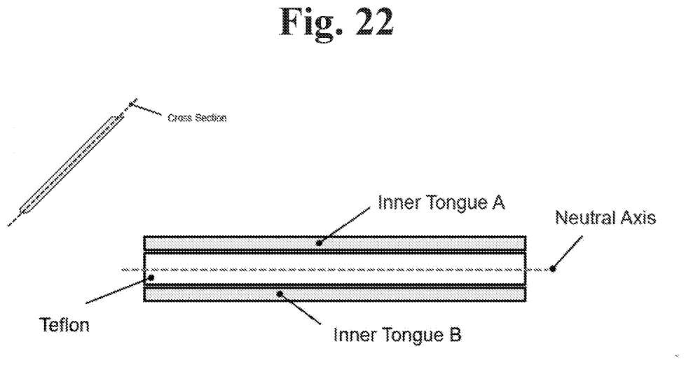

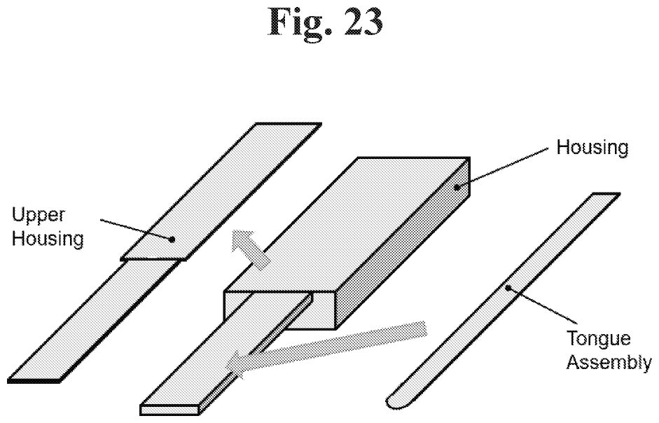

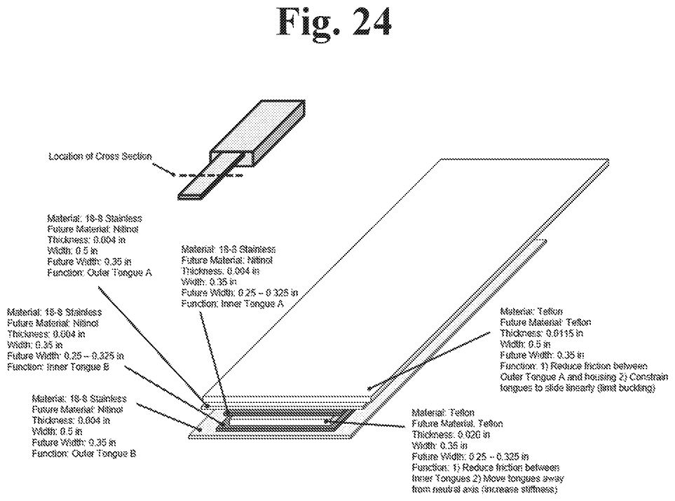

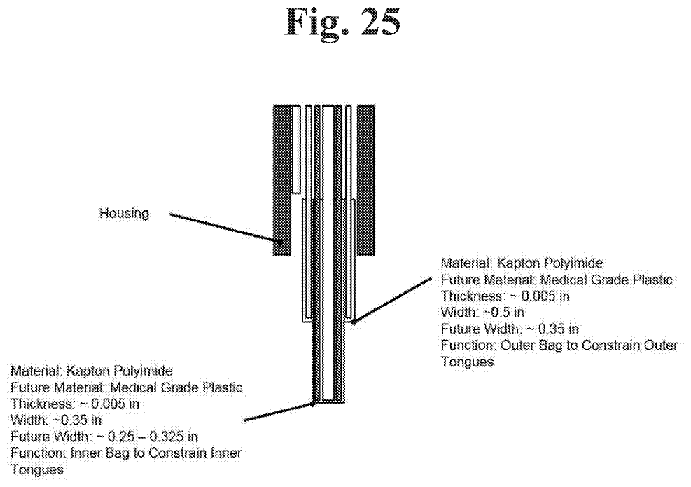

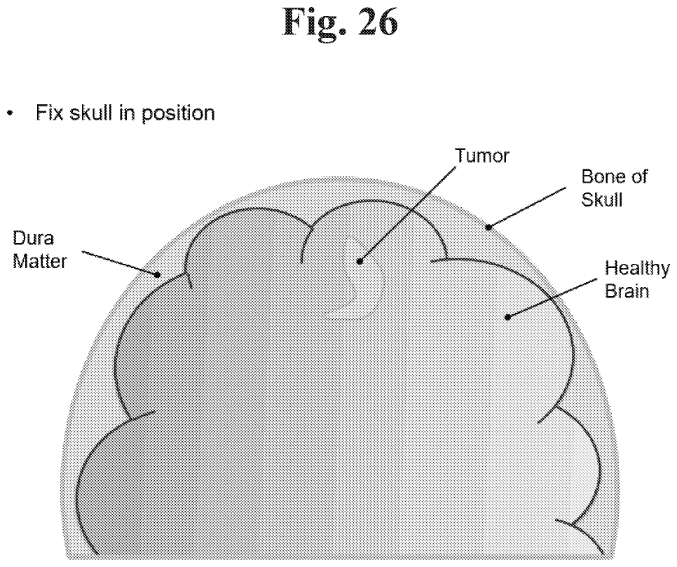

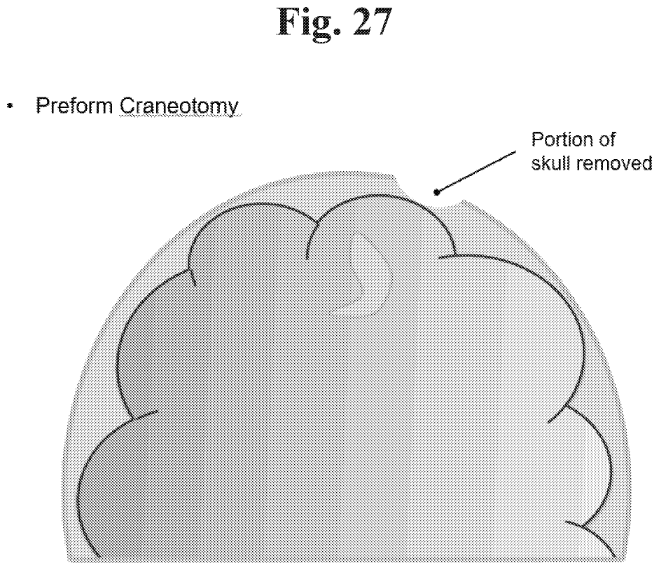

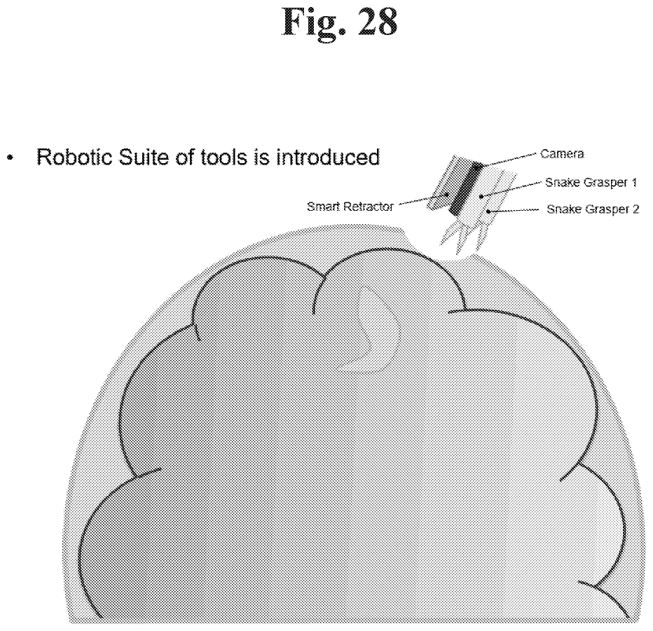

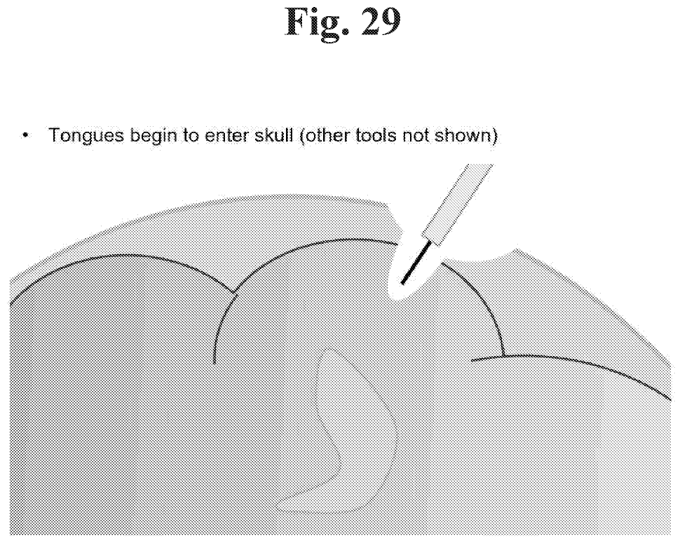

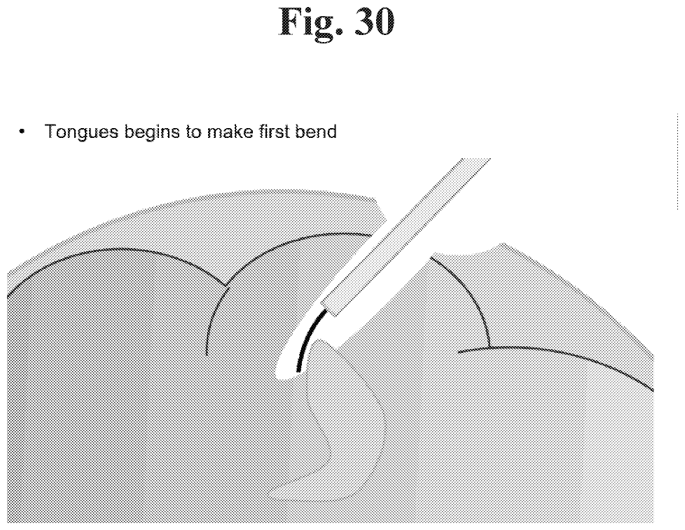

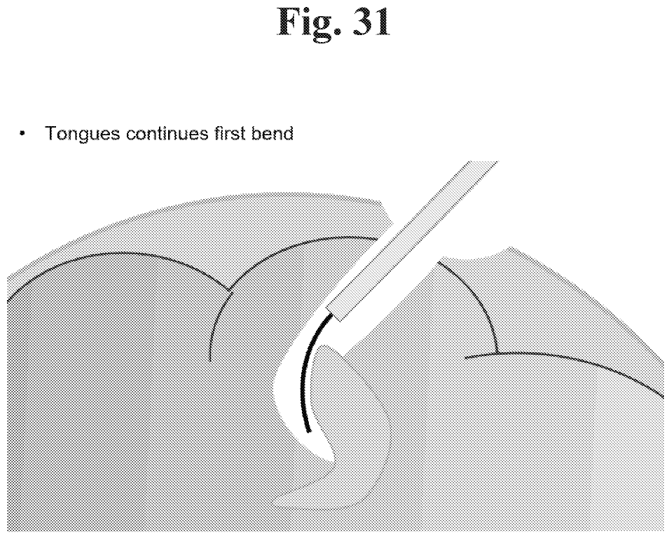

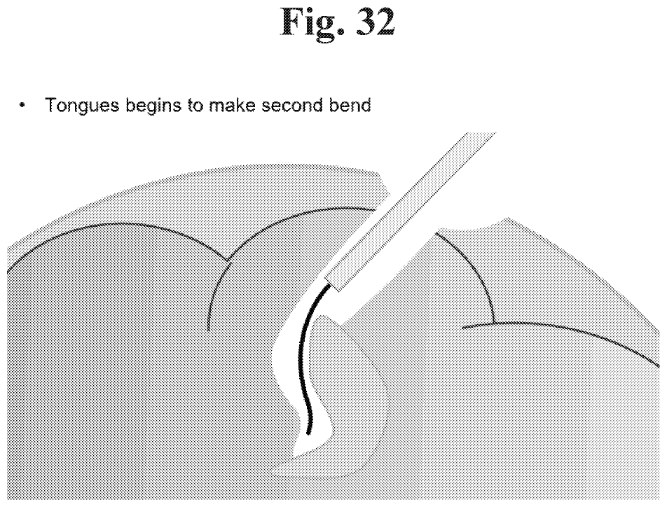

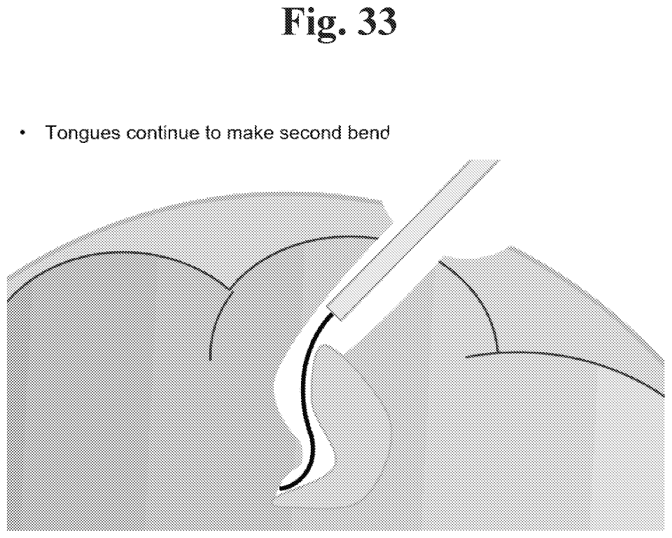

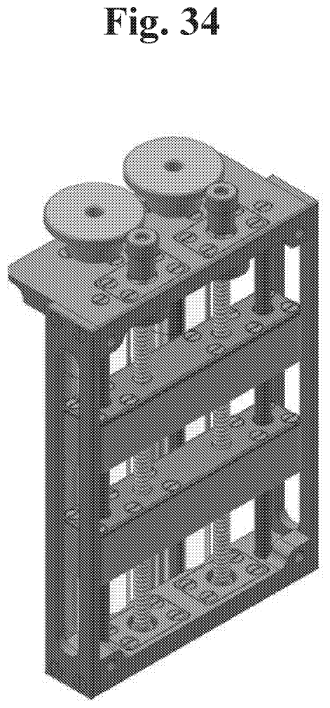

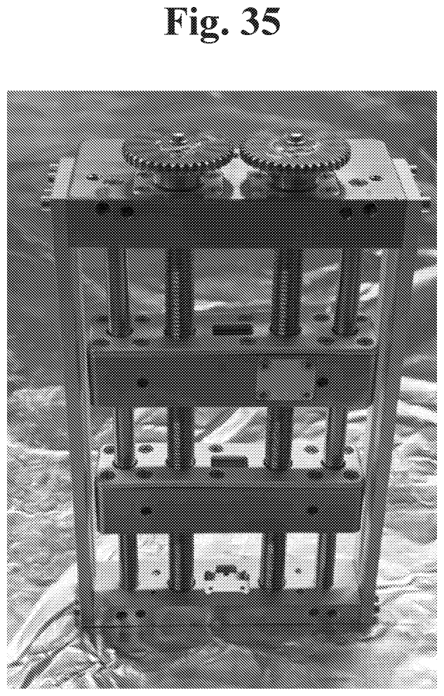

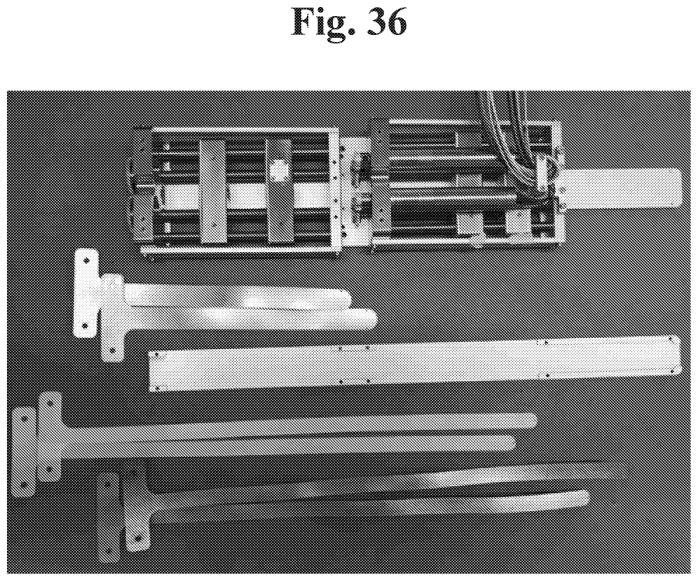

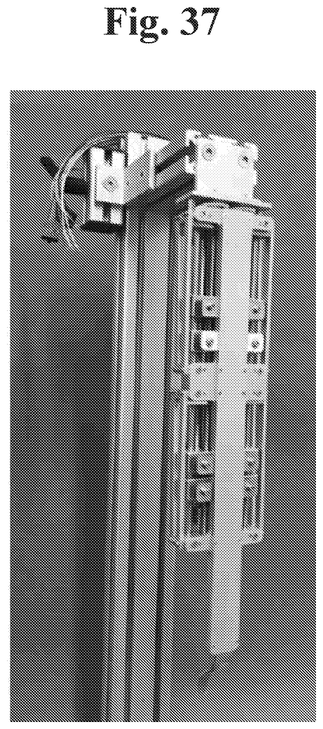

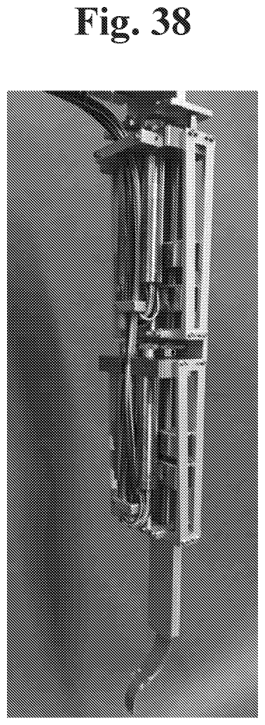

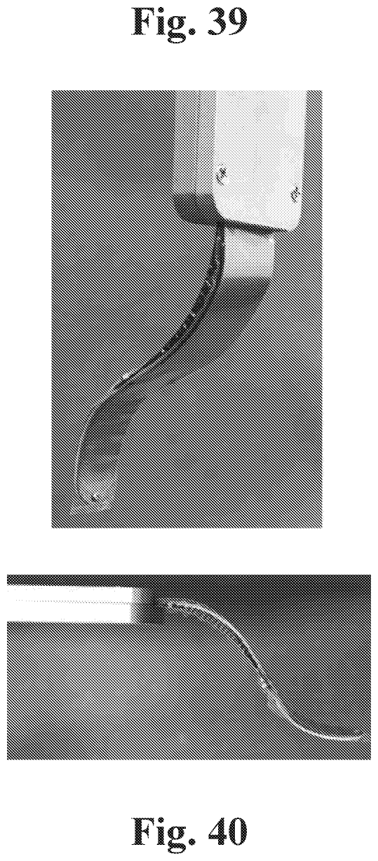

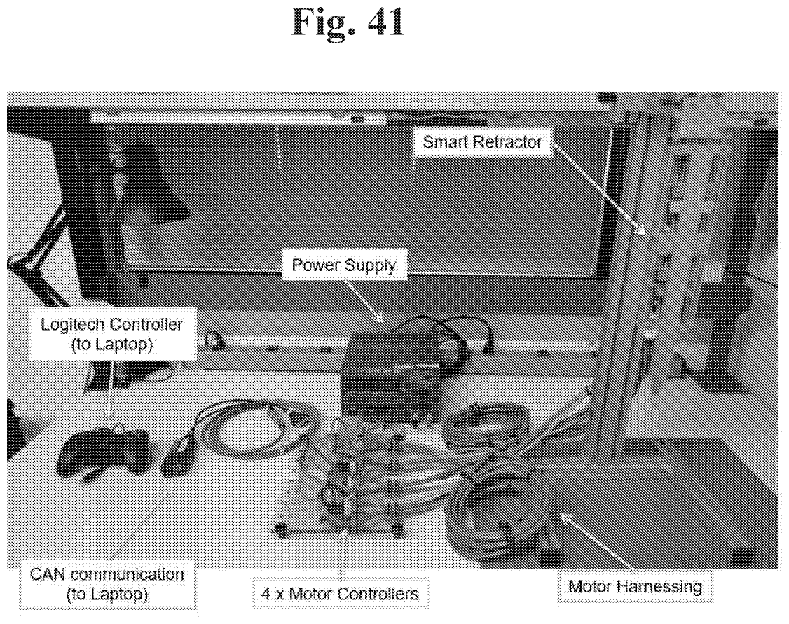

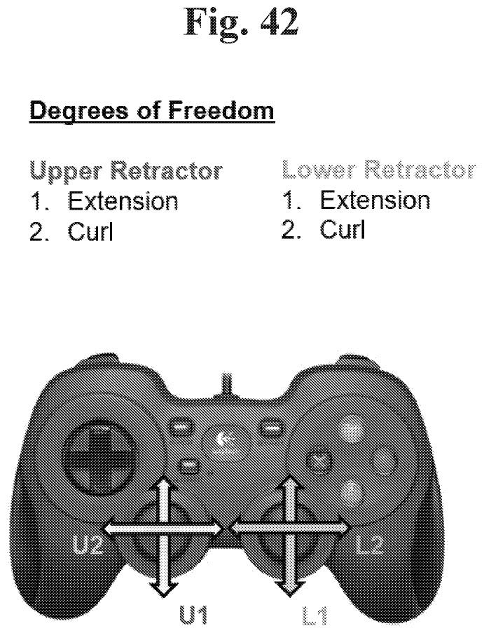

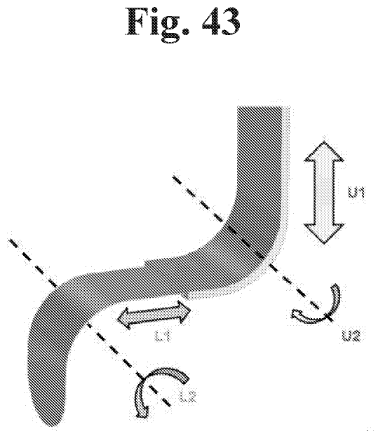

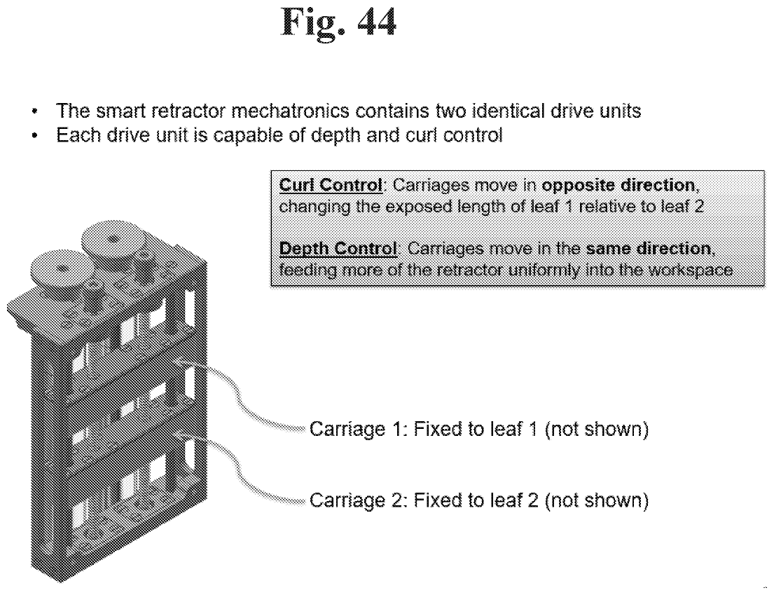

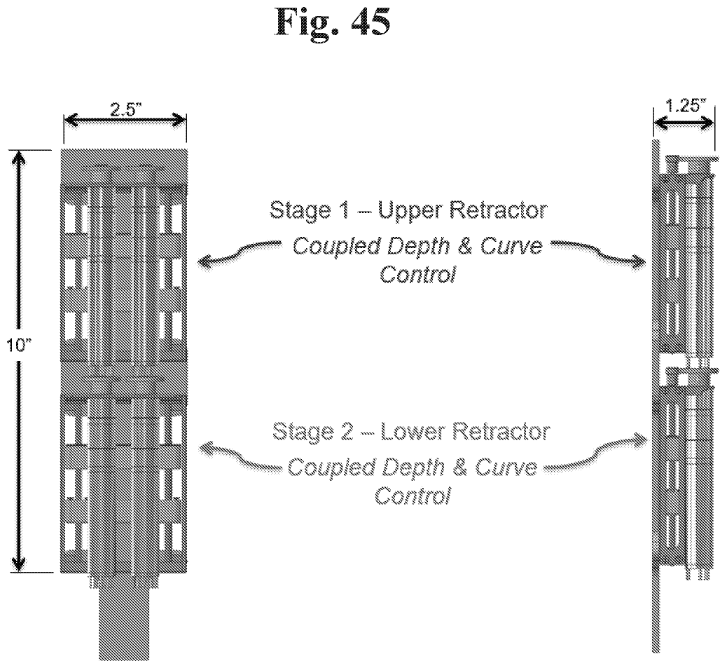

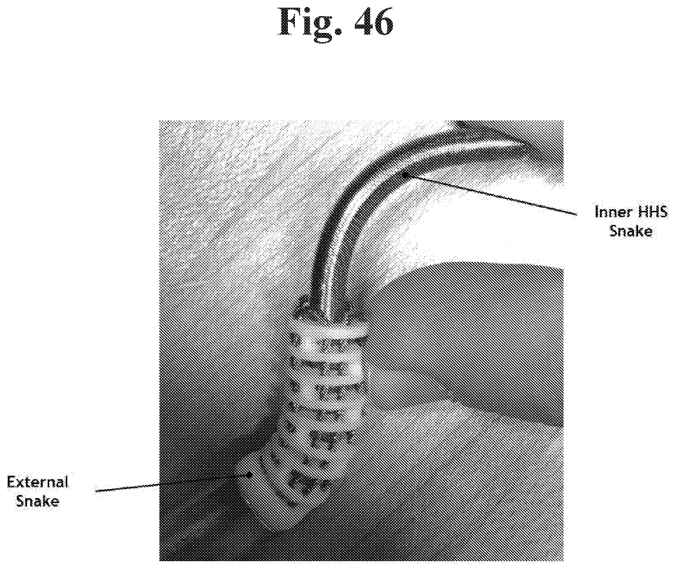

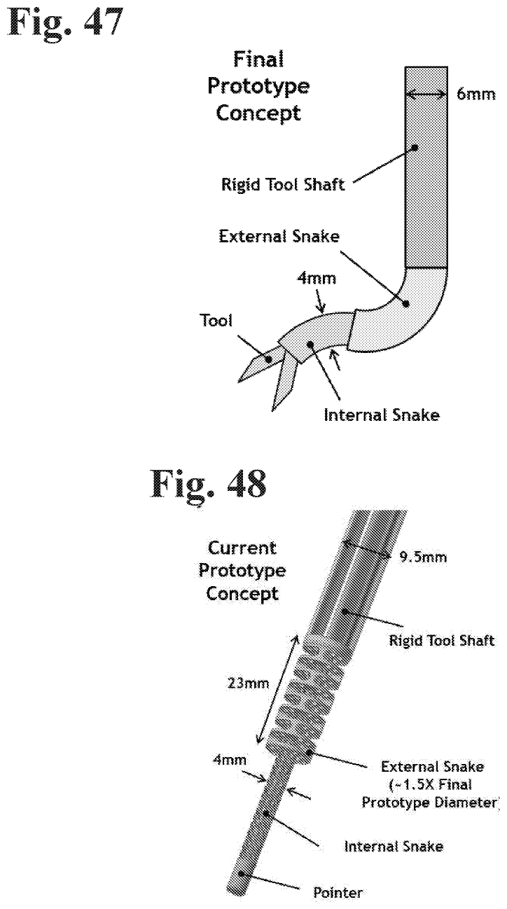

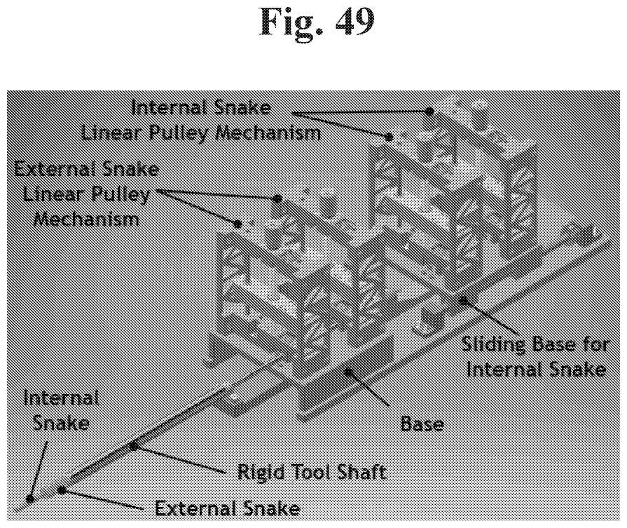

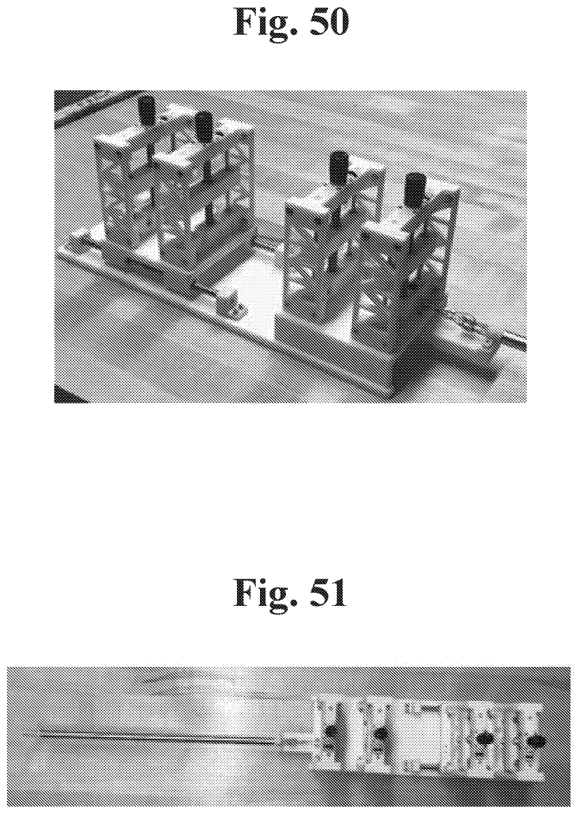

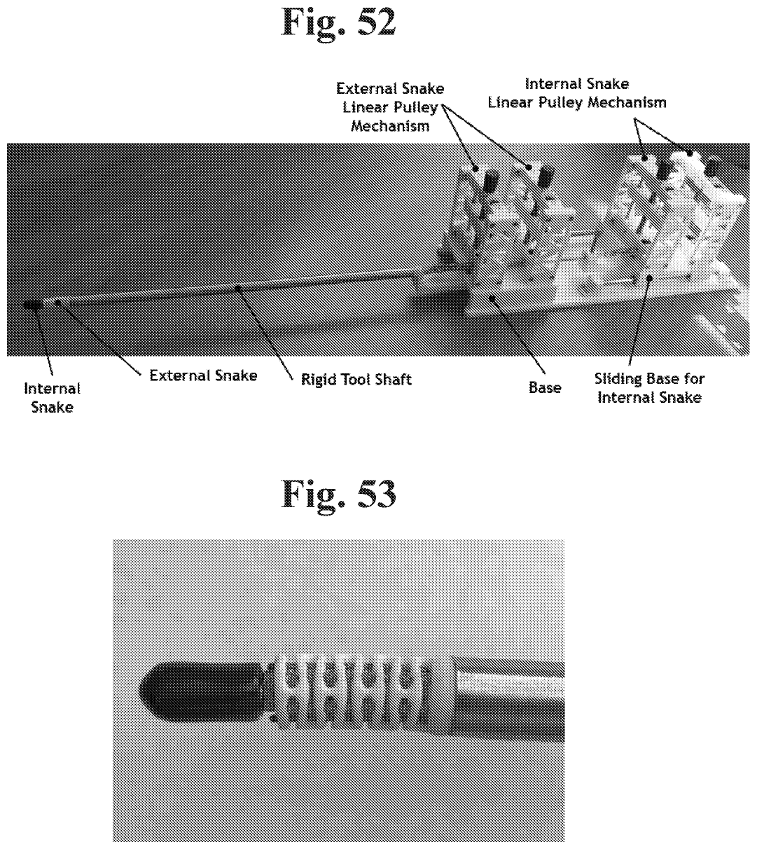

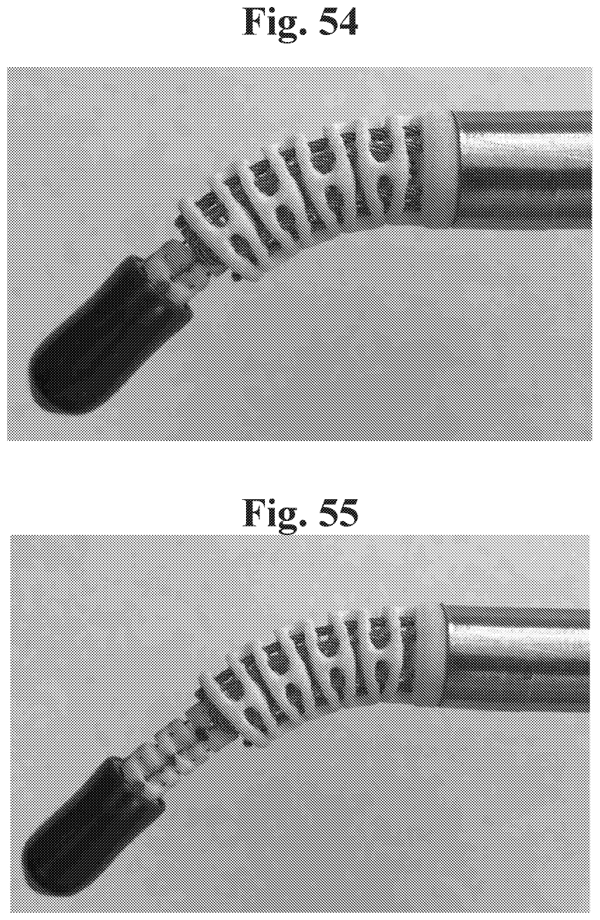

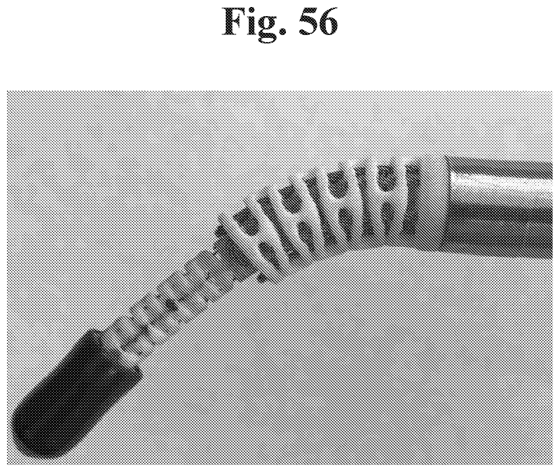

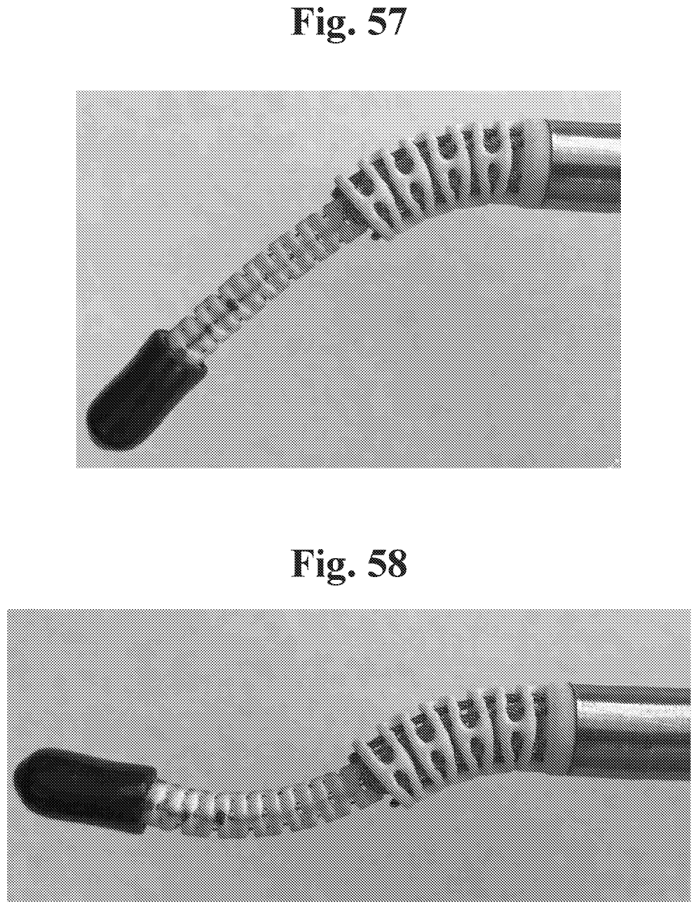

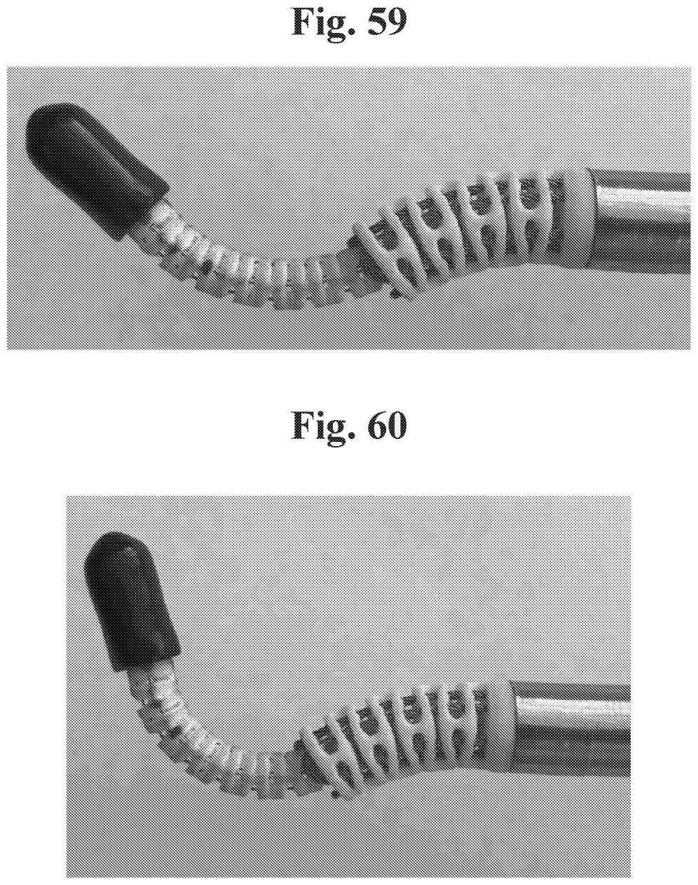

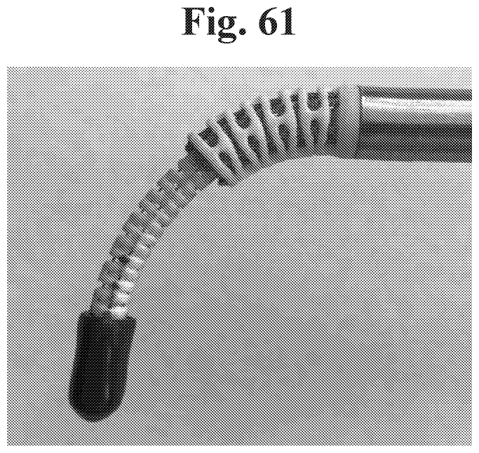

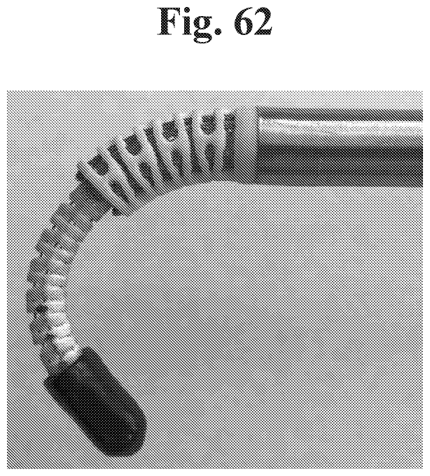

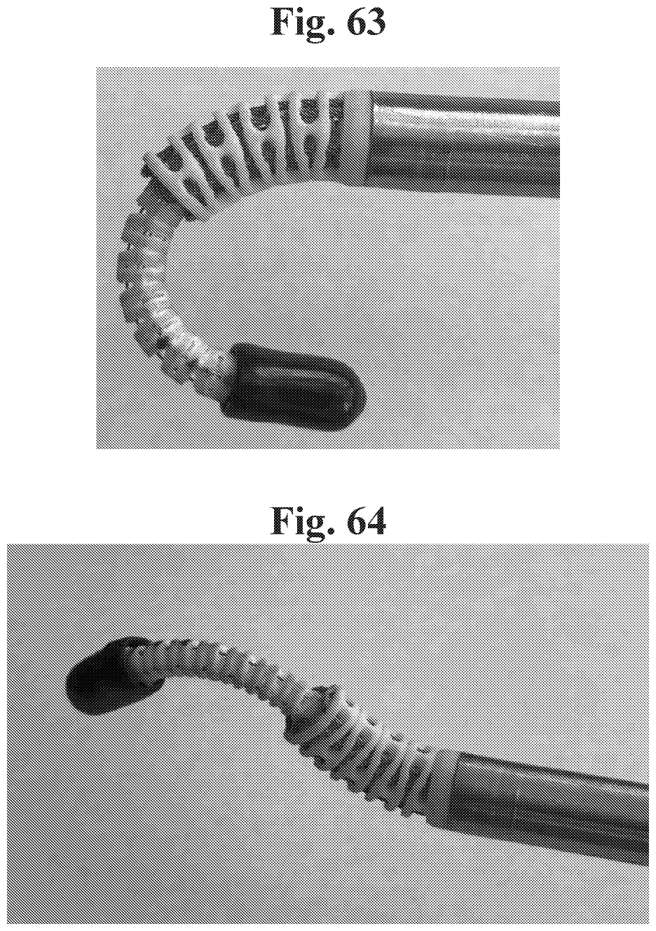

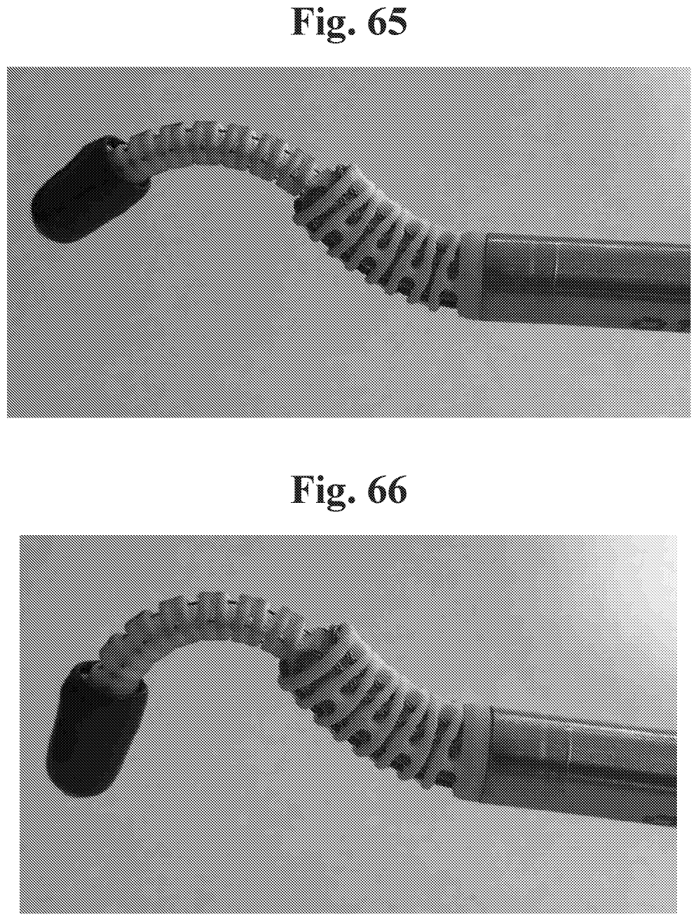

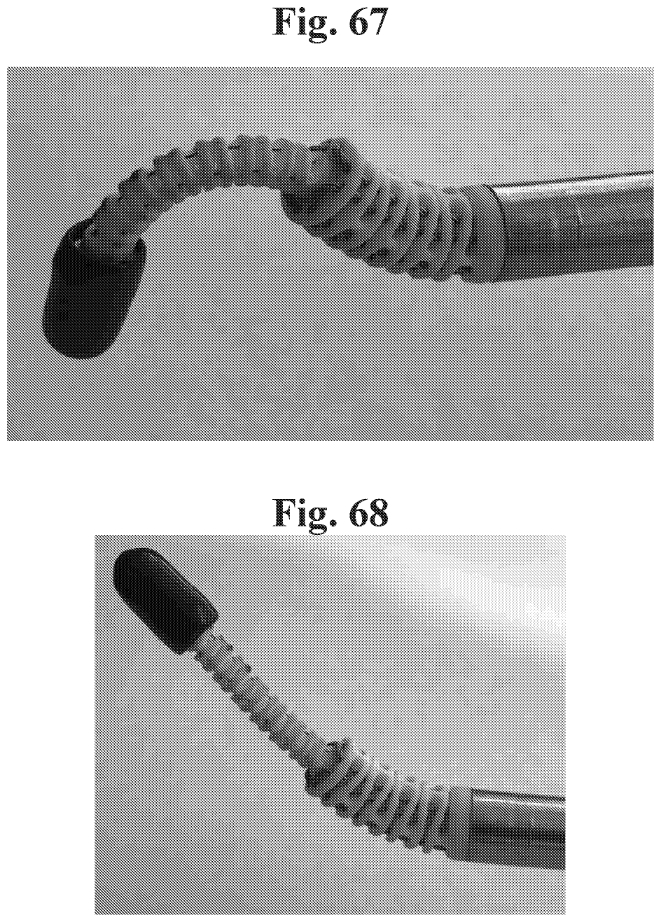

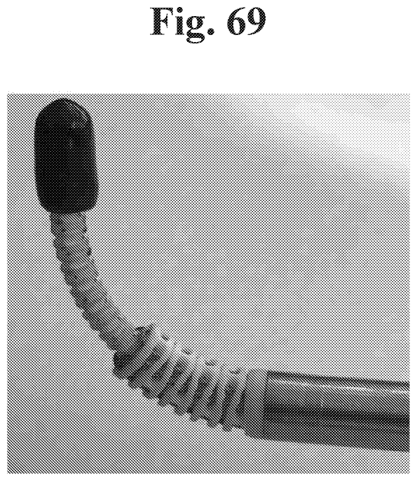

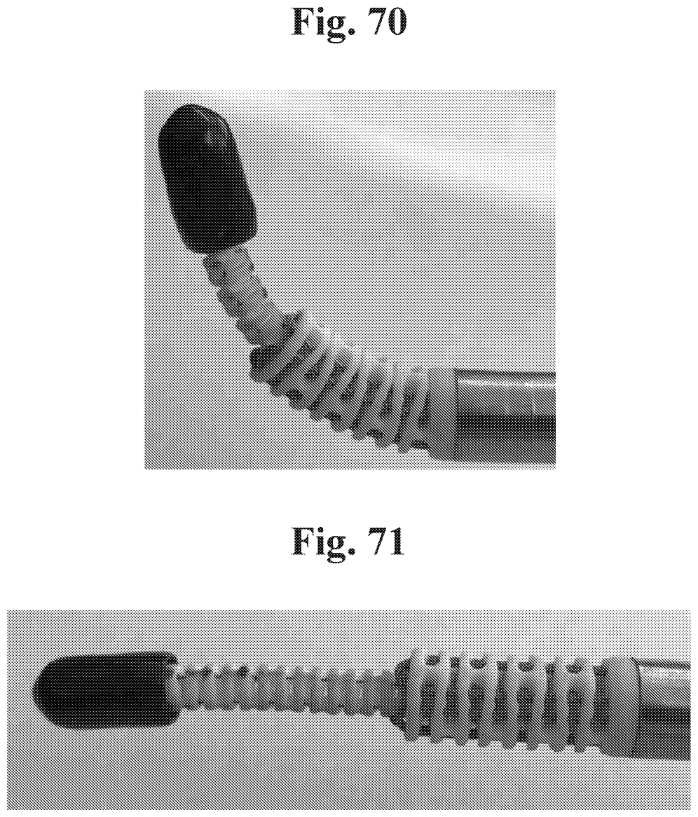

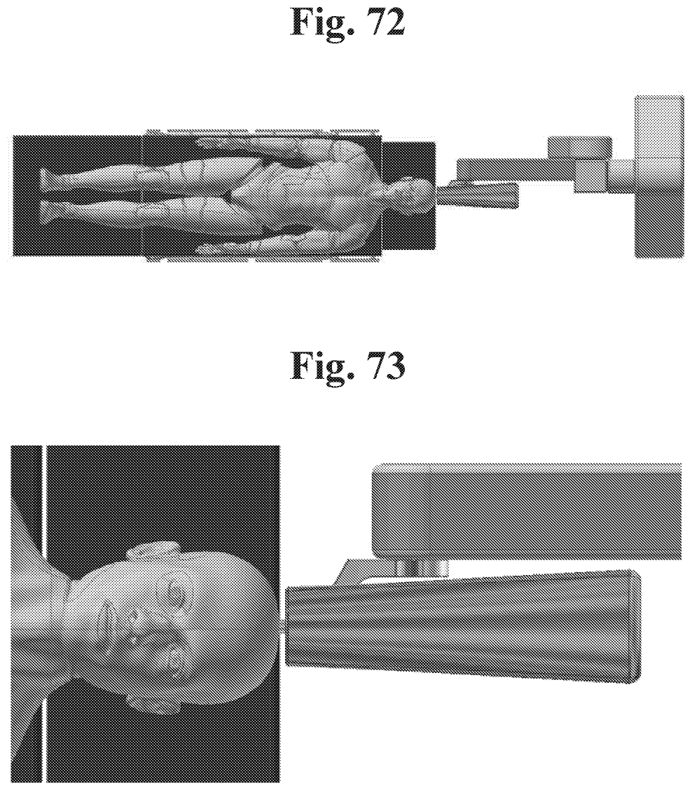

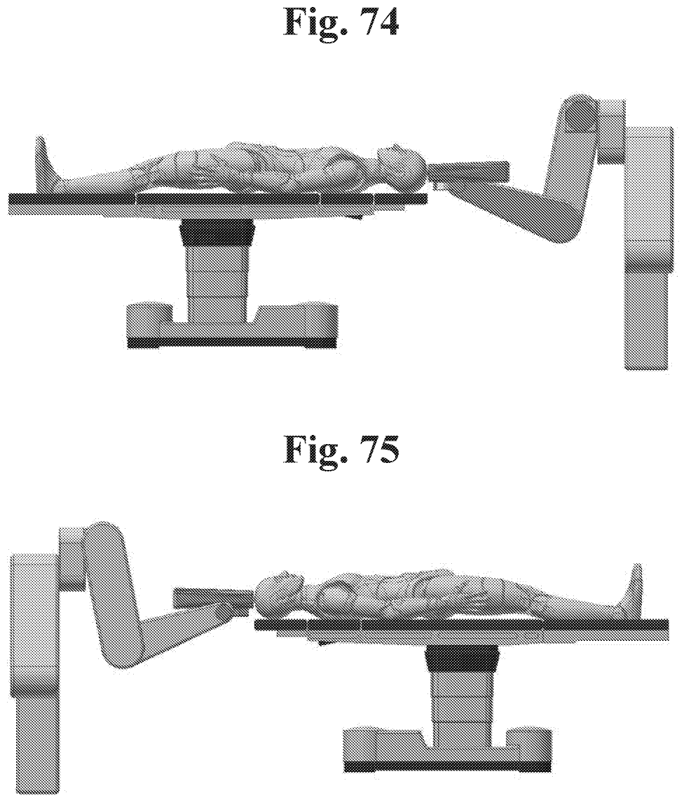

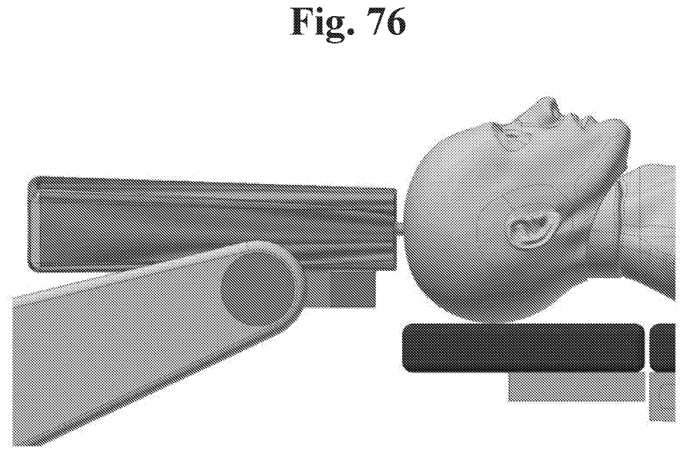

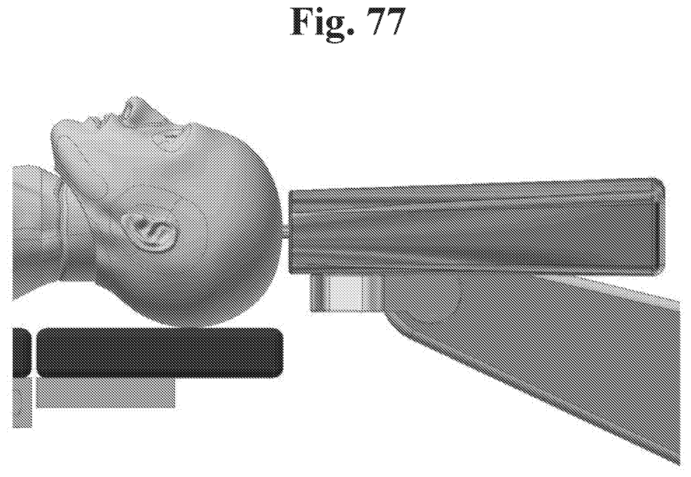

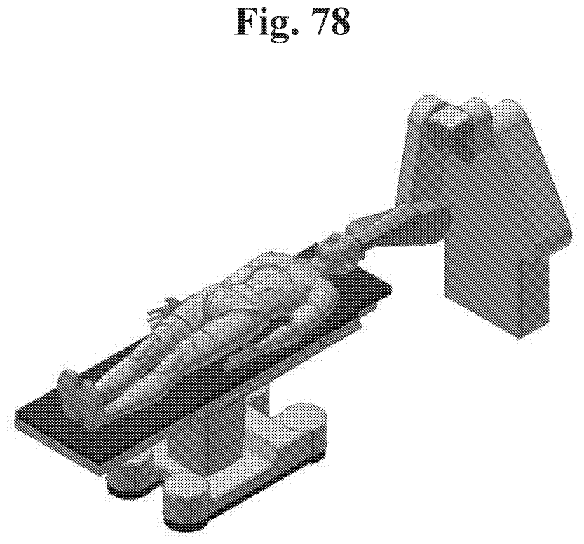

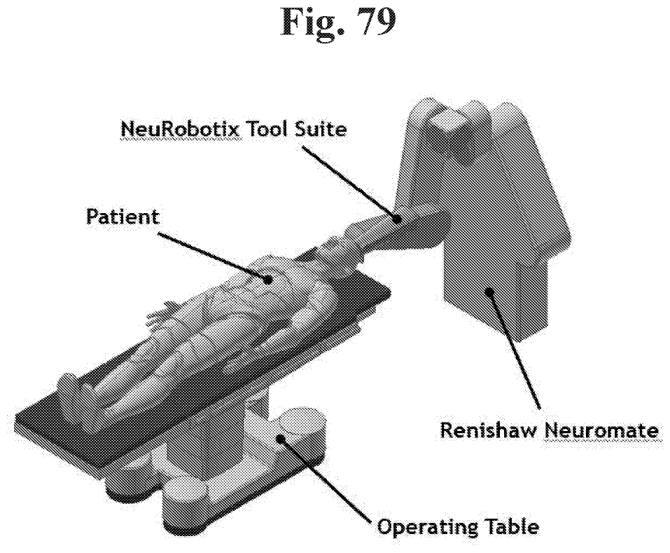

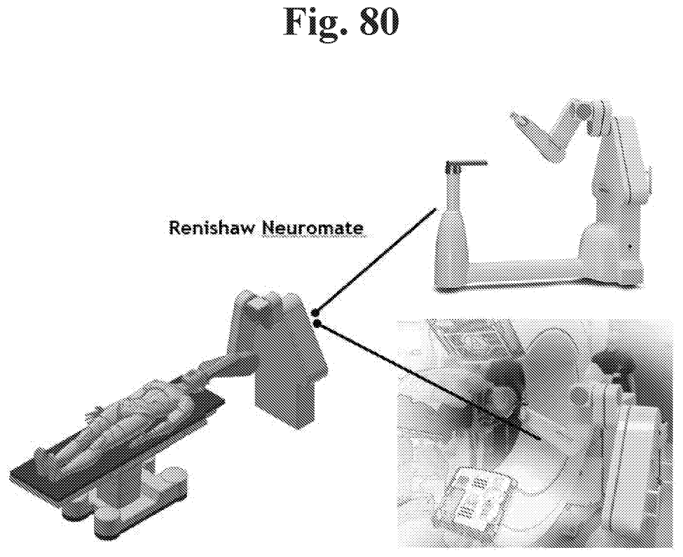

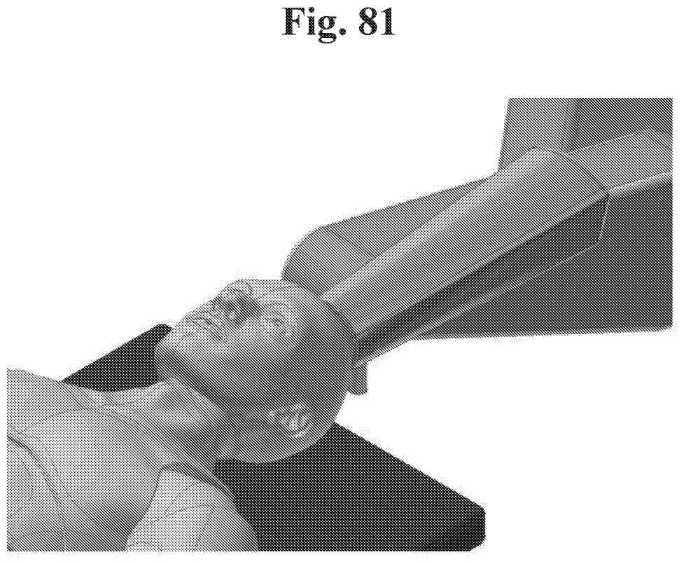

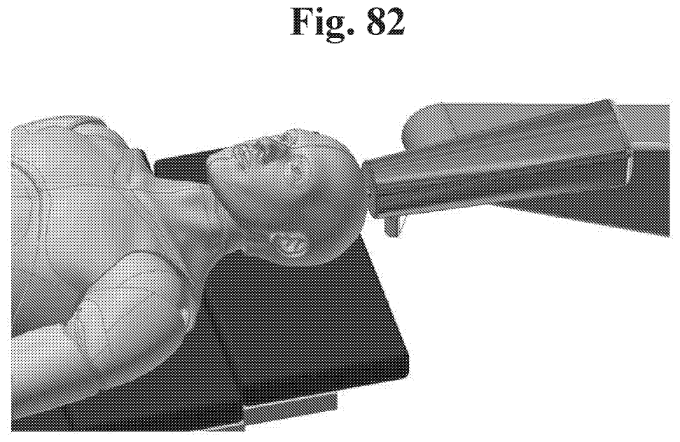

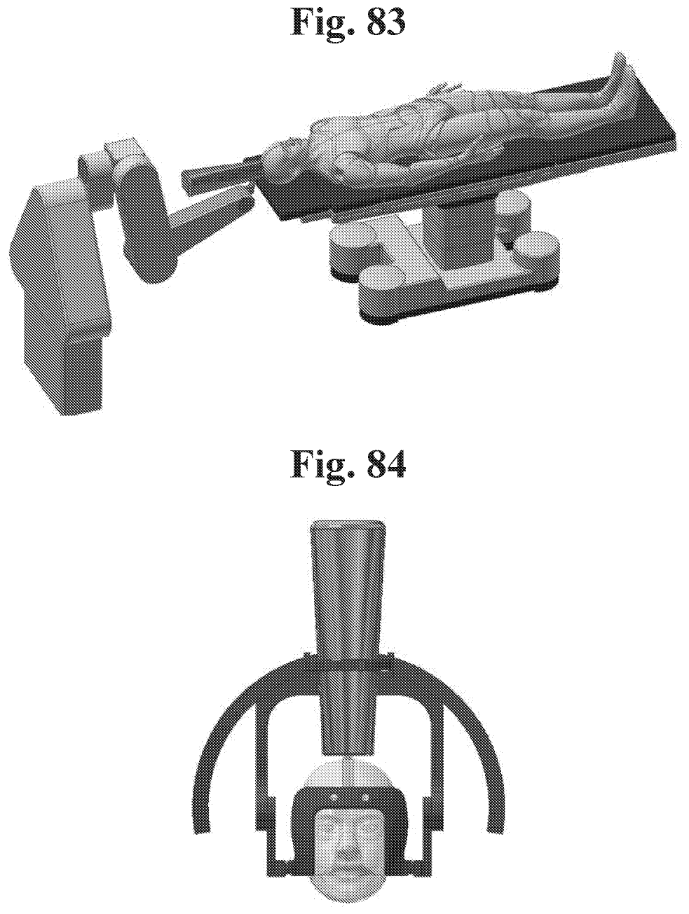

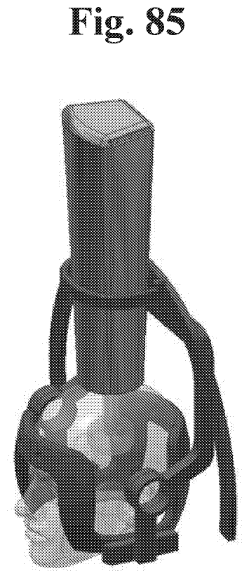

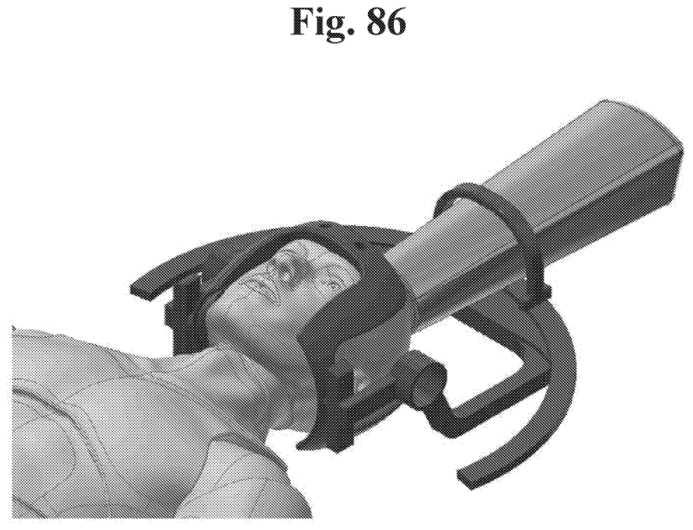

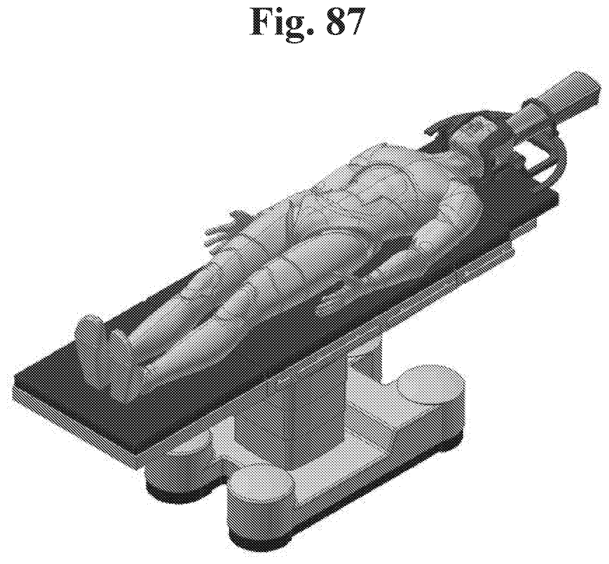

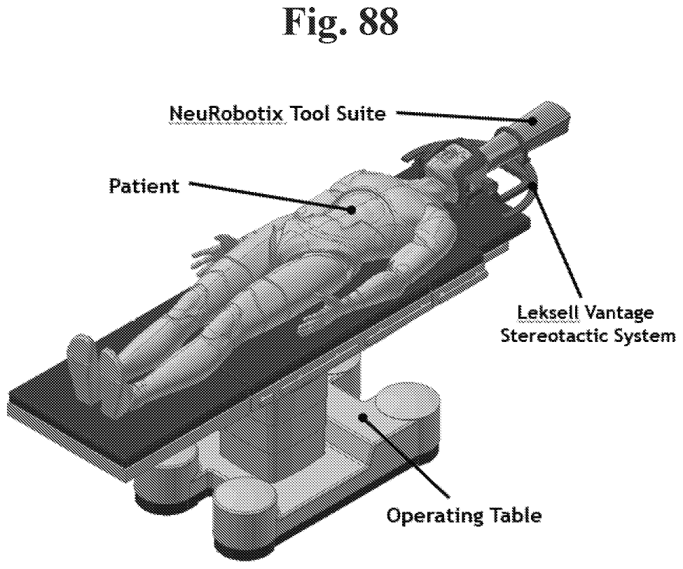

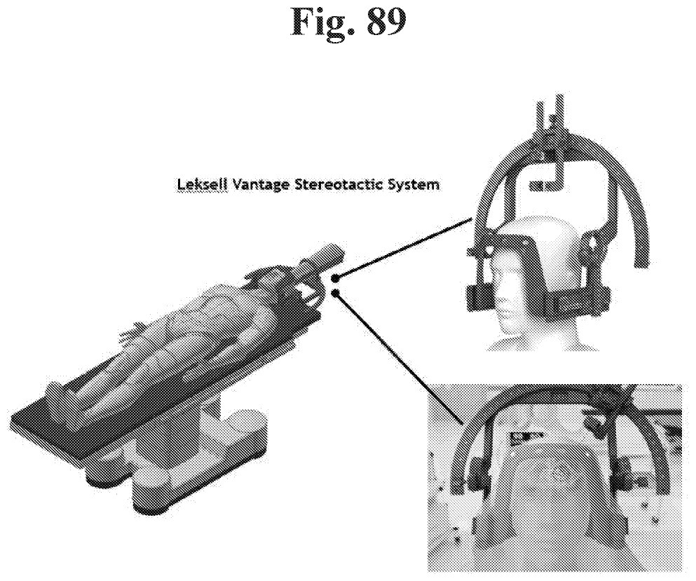

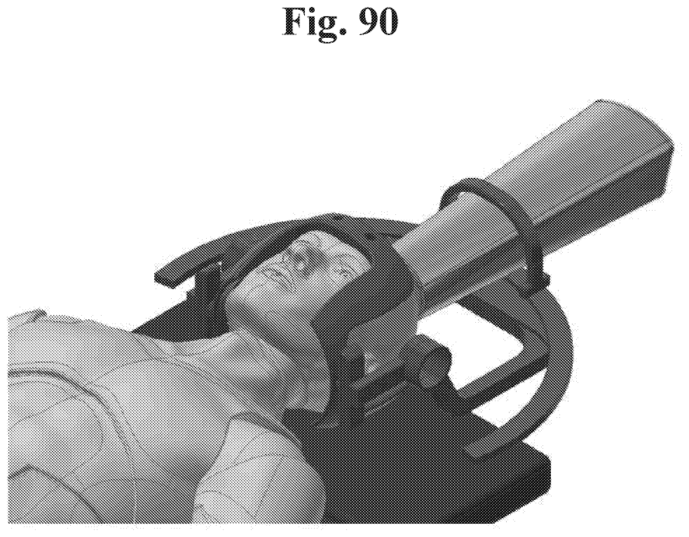

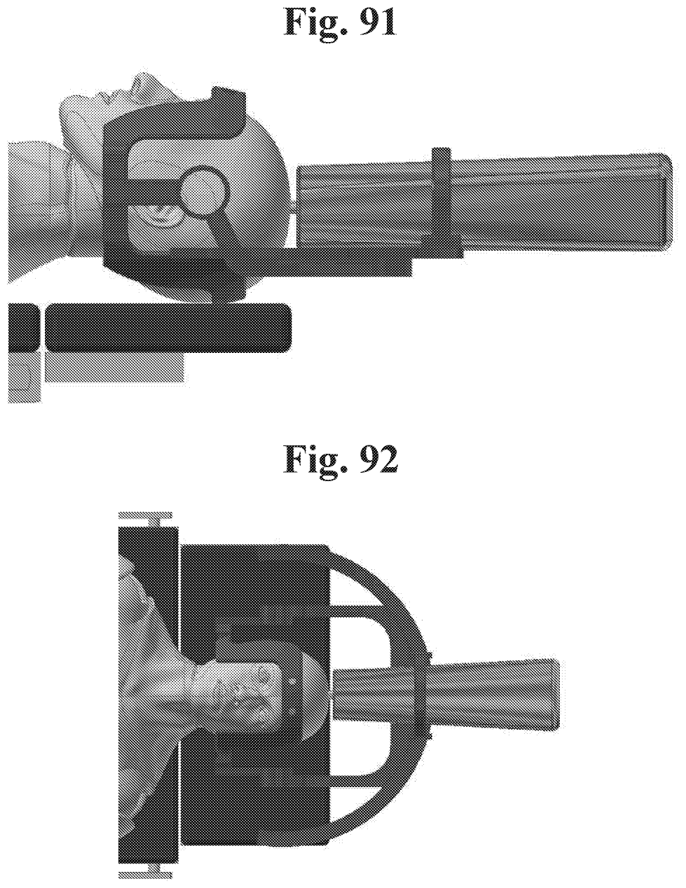

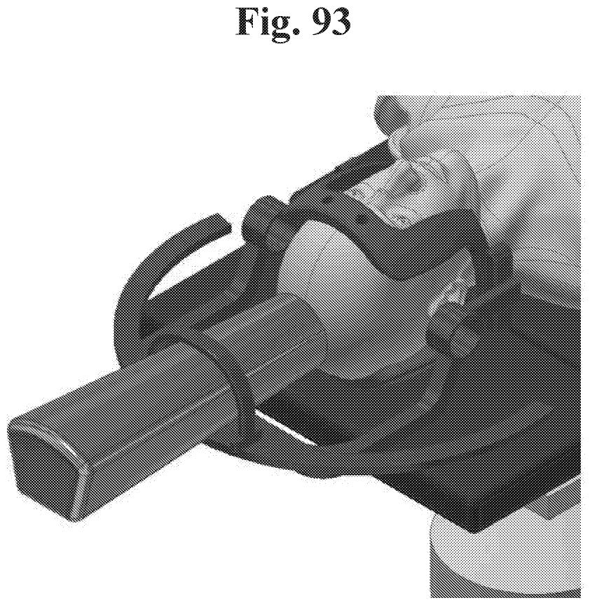

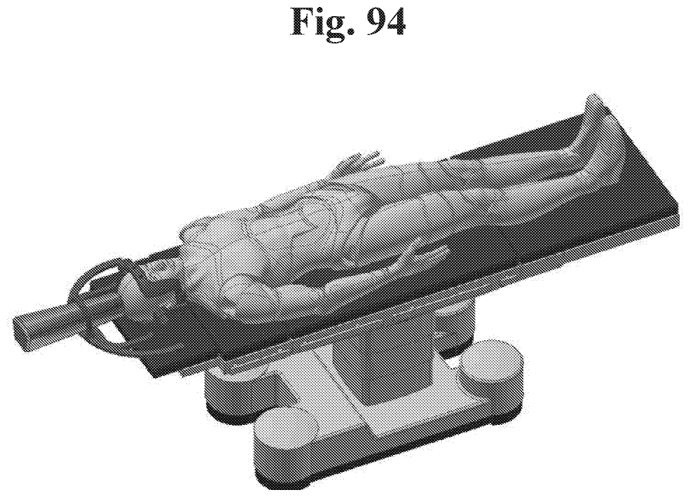

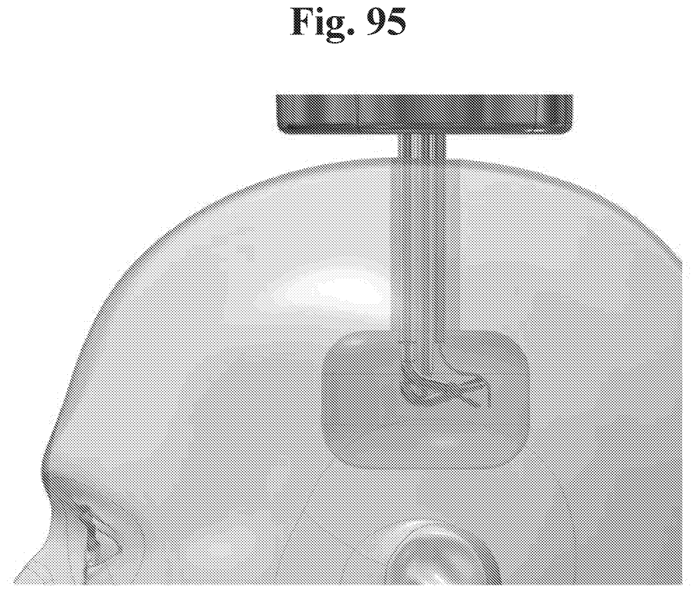

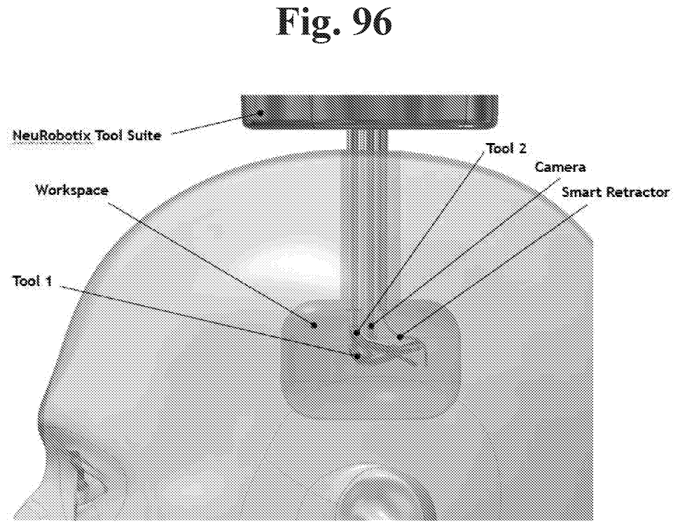

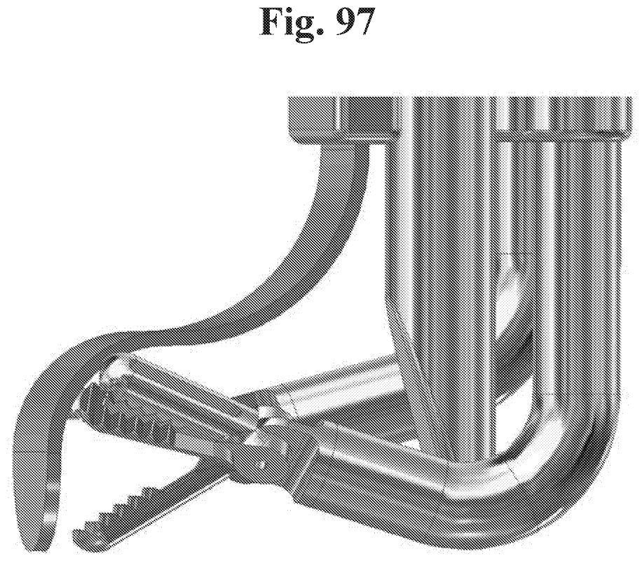

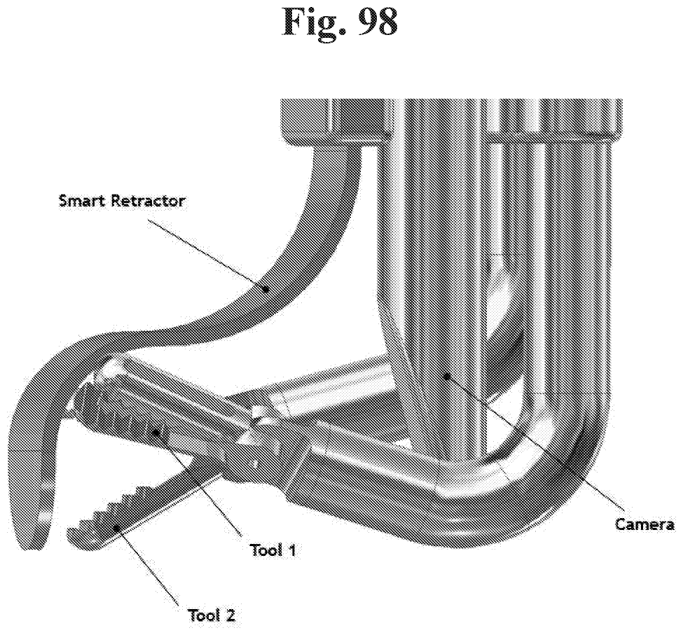

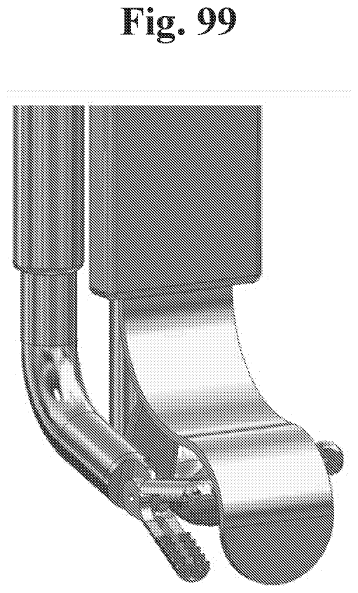

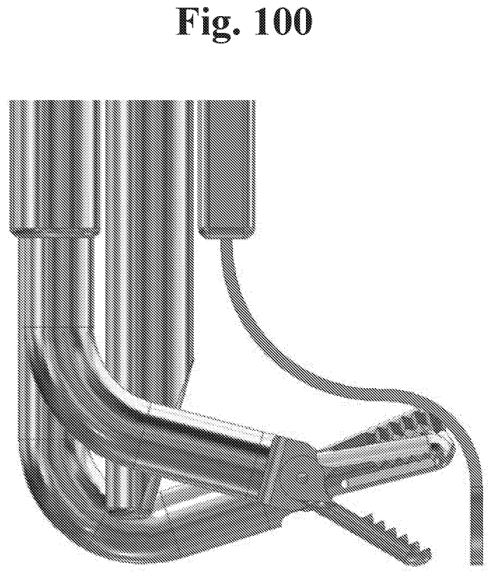

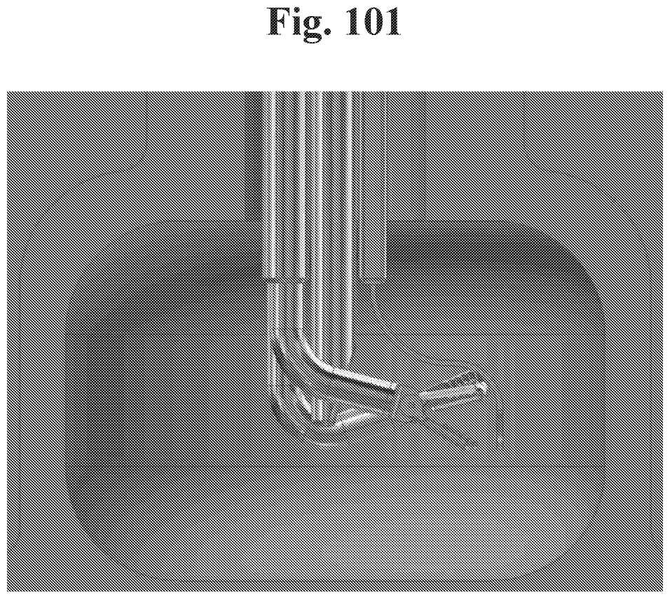

Actuations 35, 36, and 37 all function properly and in conjunction with outer sleeve 38 and inner sleeve 39 (FIG. 14). These sleeves constrain the flexing of inner and outer tongue assemblies. Inner sleeve 39 is positioned between the outer surfaces of tongue strips 15 and 16 and the inner surfaces of tongue strips 20 and 21. Outer sleeve 38 has a small incision that allows inner sleeve 39 to move freely in and out.

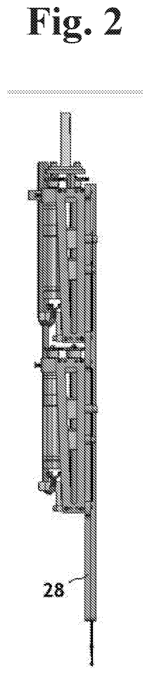

Proper constraining of tongue strips 15, 16, 20, 21 and Teflon strip 17 is accomplished with structural elements 27 and 28 (FIGS. 1 and 2). These close-fitting elements create a channel that does not allow the tongue strips to flex until they exit the channel.

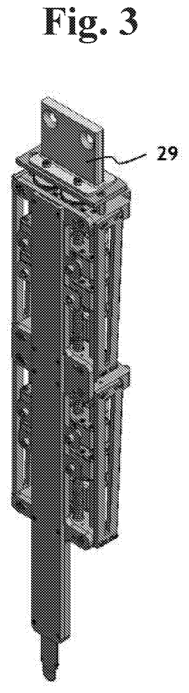

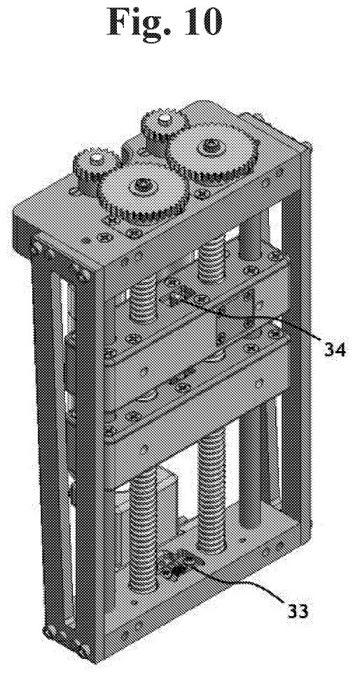

Sensing the position of carriages 14a and 14b of the upper and lower drive units 1 and 2 is accomplished with sensors 30, 33, and 40 (FIGS. 9 and 10), that get triggered by magnets 31, 32, and 34 respectively (FIGS. 9 and 10). Structural element 39 (FIG. 3) allows the entire robot to be attached to a mating fixture.

The other Figs. annexed to this specification will permit one skilled in the art to both understand and appreciate the many taught aspects of this invention. While one or more embodiments of the invention are disclosed in this patent application, other embodiments will be apparent to one skilled in the art and are contemplated as coming within the lawful scope of the invention.

What is claimed is:

1. A robotically-assisted surgical system, method and apparatus capable of achieving access to areas of the brain in addition to and outside direct line of sight through a relatively small skull access port, comprising:
a console spaced from a patient adapted to accommodate a surgeon,
robotics interconnecting the surgeon and surgical tools, said tools capable of entering and operating through a relatively small (20 mm) port in the patient's skull,
camera means comprising part of said surgical tools being interconnected to a monitor viewable by the surgeon, and
means controllable by the surgeon for gaining access to and manipulating portions of the patient's brain both within a direct line of sight and outside of said direct line of sight.
